SEQUENCE LISTING (15 sequences)

SEQ ID NO: 1 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 2 - RNA, 744 nt, Homo sapiens
  source 1..744: mol_type = mRNA
atgactatcctcttcctcacaatggtcatcagctacttcggatgtatgaaagctgcacctatgaaggaagccaacattagaggacaaggaggactcgcctatcctggagtcagaactcacggtactctggagagcgtcaatggtccaaaggctggaagcagaggtctgaccagcctggctgataccttcgagcacgttatcgaggaactcctggatgaggaccagaaggttaggccaaatgaagagaacaacaaagatgctgacctgtacacttctcgcgtgatgctcagctctcaagttccactggagcctccactcctgttcctcctggaggaatacaagaactacctggatgctgccaacatgtccatgagagttagaaggcactccgatccagctcgcagaggtgaactctctgtctgcgatagcatctctgagtgggtcaccgctgctgacaagaagacagctgtggacatgagcggtggaacagtgaccgtgctggagaaggtcccagtctctaagggtcaactgaagcagtacttctacgaaacaaagtgcaacccaatgggctatactaaggagggttgtagaggtattgataagagacattggaacagccagtgcagaacaactcaatcttatgttagggctctgactatggactccaagaagaggattggttggaggttcatcaggatcgacacttcctgcgtgtgtacactgacaatcaagagaggacgctag

SEQ ID NO: 3 - protein, 257 aa, Homo sapiens
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 4 - RNA, 774 nt, Homo sapiens
  source 1..774: mol_type = mRNA
atgtctatcctgttctatgtgatcttcctcgcatacctgagaggaatccaaggcaacaacatggatcaacgcagcctgccagaagacagcctgaactctctgatcatcaagctcatccaagcagacattctgaagaacaagctgtccaaacagatggtggatgtcaaagagaactatcagtccactctgcccaaagcagaggctcccagggagccagaaagaggtggacctgccaagtctgctttccaaccagtgattgcaatggacactgagctgctcaggcagcagaggaggtacaattctcccagggttctgctctctgattccactcctctggaaccaccacctctctacctgatggaggattacgtgggttctccagtggtggcaaacagaacaagcagacgcaagaggtatgcagaacacaaatcccacagaggtgagtatagcgtgtgcgactctgaatctctgtgggtgactgacaaatcctctgctattgacattagaggacatcaggtgacagtgctcggagagatcaagactggcaatagcccagtcaaacaatacttctacgaaactcgctgtaaagaggcaaggccagttaagaacggttgtagaggtatcgacgacaaacactggaatagccagtgtaagacttctcaaacctacgttagagccctcacatctgagaacaacaagctcgtcggatggaggtggatcagaattgatacctcctgcgtctgtgcactgagcaggaagattggaagaacctaa

SEQ ID NO: 5 - DNA, 741 bp, Homo sapiens
  source 1..741: mol_type = other DNA
atgactatcctcttcctcacaatggtcatcagctacttcggatgtatgaaagctgcacctatgaaggaagccaacattagaggacaaggaggactcgcctatcctggagtcagaactcacggtactctggagagcgtcaatggtccaaaggctggaagcagaggtctgaccagcctggctgataccttcgagcacgttatcgaggaactcctggatgaggaccagaaggttaggccaaatgaagagaacaacaaagatgctgacctgtacacttctcgcgtgatgctcagctctcaagttccactggagcctccactcctgttcctcctggaggaatacaagaactacctggatgctgccaacatgtccatgagagttagaaggcactccgatccagctcgcagaggtgaactctctgtctgcgatagcatctctgagtgggtcaccgctgctgacaagaagacagctgtggacatgagcggtggaacagtgaccgtgctggagaaggtcccagtctctaagggtcaactgaagcagtacttctacgaaacaaagtgcaacccaatgggctatactaaggagggttgtagaggtattgataagagacattggaacagccagtgcagaacaactcaatcttatgttagggctctgactatggactccaagaagaggattggttggaggttcatcaggatcgacacttcctgcgtgtgtacactgacaatcaagagaggacgc

SEQ ID NO: 6 - DNA, 771 bp, Homo sapiens
  source 1..771: mol_type = other DNA
atgtctatcctgttctatgtgatcttcctcgcatacctgagaggaatccaaggcaacaacatggatcaacgcagcctgccagaagacagcctgaactctctgatcatcaagctcatccaagcagacattctgaagaacaagctgtccaaacagatggtggatgtcaaagagaactatcagtccactctgcccaaagcagaggctcccagggagccagaaagaggtggacctgccaagtctgctttccaaccagtgattgcaatggacactgagctgctcaggcagcagaggaggtacaattctcccagggttctgctctctgattccactcctctggaaccaccacctctctacctgatggaggattacgtgggttctccagtggtggcaaacagaacaagcagacgcaagaggtatgcagaacacaaatcccacagaggtgagtatagcgtgtgcgactctgaatctctgtgggtgactgacaaatcctctgctattgacattagaggacatcaggtgacagtgctcggagagatcaagactggcaatagcccagtcaaacaatacttctacgaaactcgctgtaaagaggcaaggccagttaagaacggttgtagaggtatcgacgacaaacactggaatagccagtgtaagacttctcaaacctacgttagagccctcacatctgagaacaacaagctcgtcggatggaggtggatcagaattgatacctcctgcgtctgtgcactgagcaggaagattggaagaacc

SEQ ID NO: 7 - DNA, 633 bp, Homo sapiens
  source 1..633: mol_type = other DNA
aattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatc

SEQ ID NO: 8 - DNA, 576 bp, Homo sapiens
  source 1..576: mol_type = other DNA
cgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 9 - DNA, 269 bp, Bos taurus
  source 1..269: mol_type = other DNA
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 10 - DNA, 3024 bp, synthetic construct
  source 1..3024: mol_type = other DNA
aattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgcggccgcgccaccatgactatcctcttcctcacaatggtcatcagctacttcggatgtatgaaagctgcacctatgaaggaagccaacattagaggacaaggaggactcgcctatcctggagtcagaactcacggtactctggagagcgtcaatggtccaaaggctggaagcagaggtctgaccagcctggctgataccttcgagcacgttatcgaggaactcctggatgaggaccagaaggttaggccaaatgaagagaacaacaaagatgctgacctgtacacttctcgcgtgatgctcagctctcaagttccactggagcctccactcctgttcctcctggaggaatacaagaactacctggatgctgccaacatgtccatgagagttagaaggcactccgatccagctcgcagaggtgaactctctgtctgcgatagcatctctgagtgggtcaccgctgctgacaagaagacagctgtggacatgagcggtggaacagtgaccgtgctggagaaggtcccagtctctaagggtcaactgaagcagtacttctacgaaacaaagtgcaacccaatgggctatactaaggagggttgtagaggtattgataagagacattggaacagccagtgcagaacaactcaatcttatgttagggctctgactatggactccaagaagaggattggttggaggttcatcaggatcgacacttcctgcgtgtgtacactgacaatcaagagaggacgctagcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaagatttaaatcaccatgtctatcctgttctatgtgatcttcctcgcatacctgagaggaatccaaggcaacaacatggatcaacgcagcctgccagaagacagcctgaactctctgatcatcaagctcatccaagcagacattctgaagaacaagctgtccaaacagatggtggatgtcaaagagaactatcagtccactctgcccaaagcagaggctcccagggagccagaaagaggtggacctgccaagtctgctttccaaccagtgattgcaatggacactgagctgctcaggcagcagaggaggtacaattctcccagggttctgctctctgattccactcctctggaaccaccacctctctacctgatggaggattacgtgggttctccagtggtggcaaacagaacaagcagacgcaagaggtatgcagaacacaaatcccacagaggtgagtatagcgtgtgcgactctgaatctctgtgggtgactgacaaatcctctgctattgacattagaggacatcaggtgacagtgctcggagagatcaagactggcaatagcccagtcaaacaatacttctacgaaactcgctgtaaagaggcaaggccagttaagaacggttgtagaggtatcgacgacaaacactggaatagccagtgtaagacttctcaaacctacgttagagccctcacatctgagaacaacaagctcgtcggatggaggtggatcagaattgatacctcctgcgtctgtgcactgagcaggaagattggaagaacctaacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 11 - DNA, 744 bp, Homo sapiens
  source 1..744: mol_type = genomic DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 12 - RNA, 744 nt, Homo sapiens
  source 1..744: mol_type = genomic RNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 13 - DNA, 774 bp, Homo sapiens
  source 1..774: mol_type = genomic DNA
atgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatga

SEQ ID NO: 14 - RNA, 774 nt, Homo sapiens
  source 1..774: mol_type = genomic RNA
atgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatga

SEQ ID NO: 15 - RNA, 1569 nt, Homo sapiens
  source 1..1569: mol_type = other RNA
atgactatcctcttcctcacaatggtcatcagctacttcggatgtatgaaagctgcacctatgaaggaagccaacattagaggacaaggaggactcgcctatcctggagtcagaactcacggtactctggagagcgtcaatggtccaaaggctggaagcagaggtctgaccagcctggctgataccttcgagcacgttatcgaggaactcctggatgaggaccagaaggttaggccaaatgaagagaacaacaaagatgctgacctgtacacttctcgcgtgatgctcagctctcaagttccactggagcctccactcctgttcctcctggaggaatacaagaactacctggatgctgccaacatgtccatgagagttagaaggcactccgatccagctcgcagaggtgaactctctgtctgcgatagcatctctgagtgggtcaccgctgctgacaagaagacagctgtggacatgagcggtggaacagtgaccgtgctggagaaggtcccagtctctaagggtcaactgaagcagtacttctacgaaacaaagtgcaacccaatgggctatactaaggagggttgtagaggtattgataagagacattggaacagccagtgcagaacaactcaatcttatgttagggctctgactatggactccaagaagaggattggttggaggttcatcaggatcgacacttcctgcgtgtgtacactgacaatcaagagaggacgcgagggcagaggaagtctgctaacatgcggtgacgtcgaggagaatcctggcccaatgtctatcctgttctatgtgatcttcctcgcatacctgagaggaatccaaggcaacaacatggatcaacgcagcctgccagaagacagcctgaactctctgatcatcaagctcatccaagcagacattctgaagaacaagctgtccaaacagatggtggatgtcaaagagaactatcagtccactctgcccaaagcagaggctcccagggagccagaaagaggtggacctgccaagtctgctttccaaccagtgattgcaatggacactgagctgctcaggcagcagaggaggtacaattctcccagggttctgctctctgattccactcctctggaaccaccacctctctacctgatggaggattacgtgggttctccagtggtggcaaacagaacaagcagacgcaagaggtatgcagaacacaaatcccacagaggtgagtatagcgtgtgcgactctgaatctctgtgggtgactgacaaatcctctgctattgacattagaggacatcaggtgacagtgctcggagagatcaagactggcaatagcccagtcaaacaatacttctacgaaactcgctgtaaagaggcaaggccagttaagaacggttgtagaggtatcgacgacaaacactggaatagccagtgtaagacttctcaaacctacgttagagccctcacatctgagaacaacaagctcgtcggatggaggtggatcagaattgatacctcctgcgtctgtgcactgagcaggaagattggaagaacctaa

(19) United States
(12) Patent Application Publication
(10) Pub. No.: US 2026/0355280 A1
(43) Pub. Date: Nov. 12, 2026

(54) METHOD OF PRODUCING PROTEIN

(71) Applicant: NewSouth Innovations Pty Limited, Sydney, New South Wales (AU)

(72) Inventors: Gary David Housley, Sydney, New South Wales (AU); Jeremy Pinyon, Sydney, New South Wales (AU); Gerog von Jonquieres, Sydney, New South Wales (AU); Nigel Hamilton Lovell, Sydney, New South Wales (AU)

(21) Appl. No.: 19/142,514

(22) PCT Filed: Dec. 22, 2023
(86) PCT No.: PCT/AU2023/051370

§ 371 Date: Jun. 23, 2025

(30) Foreign Application Priority Data
Dec. 22, 2022 (AU) 2022903976

(51) Int. Cl. A61K 38/18; A61K 41/00; A61K 48/00; A61N 1/04; A61N 1/08; A61N 1/32

(52) U.S. Cl. CPC A61K 38/185; A61K 41/0047; A61K 48/005; A61N 1/0416; A61N 1/0476; A61N 1/08; A61N 1/327

(57) ABSTRACT

The present disclosure relates to a method of expressing a protein of interest in one or more cells of tissue in a target region, where RNA comprising nucleotide sequence capable of expressing the protein of interest in the one of more cells of the tissue is introduced to the tissue in the target region, and entry of the RNA into cells of the tissue is promoted using electrotransfer. Also described is nucleic acid molecules for use with the method, and a kit for implementing the method.

### FIELD

The present invention relates to a method of producing recombinant protein in one or more cells of a tissue, encoded by a recombinant nucleic acid molecule, and to uses thereof, for increasing Brain-Derived Neurotrophic Factor (BDNF) and/or Neurotrophin-3 (NT-3) expression in a mammalian cell, and/or for promoting neuron growth/survival in mammalian tissue, and to a method of promoting neuron growth/survival in mammalian tissue.

### BACKGROUND

Expression of a recombinant protein of interest in tissue from an introduced nucleic acid is usually achieved through delivery of the nucleic acid via viral delivery vehicles, liposomes or nanoparticles. Production of a protein in vivo from a nucleic acid using such methods requires delivery of the nucleic acid into the appropriate cell, unpackaging of the nucleic acid, transcription (if the nucleic acid is DNA) and transport of the nucleic acid to the appropriate cellular compartment for translation, and ultimately translation of the mRNA. Using such approaches, expression of protein encoded by the nucleic acid typically takes 24 hours or greater.

Rapid expression of protein in a tissue would be advantageous, particularly in circumstances where cell survival is dependent on expression of the protein of interest. In this regard, rapid expression of protein would be advantageous in, for example, growth and survival of neural cells.

There are a variety of conditions and circumstances where neural growth and/or neural survival/neuroprotection would be beneficial, and would benefit from rapid protein production. For example, spinal cord injury, neurodegenerative disorders, deafness resulting from loss of auditory nerve fibres, transplantation to promote nerve growth and formation of neural connections, reconstructive surgery, stroke (Craig A J, Housley G D. Front Mol Neurosci. 2016 May 24; 9:34) and traumatic brain injury, neuroprotection in epilepsy (Falcicchia et al. Mol Ther Methods Clin Dev., 2018, 9:211-224), and in bionic applications to promote the neural connection between bionic devices and nerves.

Promotion of neural growth and/or neural survival would therefore be beneficial in people and animals suffering from neural degeneration, loss of neurons, or for applications where nerve growth or survival is required.

What is needed are improved approaches for rapid recombinant protein expression, and in particular rapid recombinant protein expression which promotes neural growth and/or neural survival in tissue.

### SUMMARY

The inventors have found that when mRNA molecules encoding a protein of interest, is introduced into tissue by electroporation or electrotransfer, translation of the protein occurs more rapidly and to a higher level than when the protein is expressed from DNA. Moreover, the translation is more rapid following delivery of mRNA by electrotransfer than when the mRNA is introduced using other delivery methods. In this regard, and as described in the examples, delivery of naked mRNA molecules encoding a protein into cells using electroporation results in detectable translation of the encoded protein within 3 hours. Moreover, as also described in the Examples, protein expression from delivery of naked mRNA was greater than that from DNA encoding the same protein, with maximum expression of the protein occurring within 1-3 days of delivery of the mRNA (as compared to 3-14 days for expression from DNA). This is advantageous in circumstances where rapid and high expression is required, such as in circumstances where neuron survival or growth is desired.

Accordingly, a first aspect provides a method of expressing a protein of interest in one or more cells of tissue in a target region, the method comprising:
(a) introducing to the tissue in the target region RNA comprising nucleotide sequence capable of expressing the protein of interest in the one of more cells of the tissue; and
(b) inducing via two or more electrodes proximate the target region an electric potential difference in the tissue of the target region, the electric potential difference causing electric fields through the target region, to thereby promote entry of the RNA into the one or more cells of the tissue.

A second aspect provides a method of producing a recombinant protein of interest in one or more cells of tissue in a target region, the method comprising:
(a) introducing to the tissue in the target region RNA comprising nucleotide sequence capable of expressing the protein of interest in the one of more cells of the tissue; and
(b) inducing via two or more electrodes proximate the target region an electric potential difference in the tissue of the target region, the electric potential difference causing an electric field through the target region, to thereby promote entry of the RNA into the one or more cells of the tissue,
   wherein the protein of interest is human BDNF protein and/or human NT-3 protein, and wherein the nucleotide sequence encoding the BDNF and/or NT-3 protein is codon optimised for translation of the protein in a mammalian cell at a level that is higher relative to the level of protein translation from a nucleotide sequence encoding the protein that is not codon optimised.

The inventors have further developed a nucleic acid molecule, which, when introduced into mammalian cells is efficiently translated, resulting in production and secretion of BDNF and/or NT-3 at levels that promote growth and/or survival of neurons.

A third aspect provides a nucleic acid molecule comprising a nucleotide sequence encoding human BDNF protein, wherein the nucleotide sequence is codon optimised for translation of BDNF protein in a mammalian cell at a level that is higher relative to the level of BDNF protein translation from a nucleotide sequence encoding human BDNF protein that is not codon optimised.

A fourth aspect provides a nucleic acid molecule comprising a nucleotide sequence encoding human NT-3 protein, wherein the nucleotide sequence is codon optimised for translation of NT-3 protein in a mammalian cell at a level that is higher relative to the level of NT-3 protein translation from a nucleotide sequence encoding human NT-3 protein that is not codon optimised.

A fifth aspect provides a nucleic acid molecule comprising a nucleotide sequence encoding human BDNF protein and a nucleotide sequence encoding human NT-3 protein, wherein the nucleotide sequence encoding the BDNF and/or NT-3 proteins is codon optimised for translation of the protein in a mammalian cell at a level that is higher relative to the level of protein translation from a nucleotide sequence encoding the protein that is not codon optimised.

In one embodiment of the third to fifth aspects, the nucleic acid molecule is an RNA molecule, typically an mRNA molecule.

A sixth aspect provides a vector comprising the nucleic acid molecule of any one of third to fifth aspects.

In one embodiment of the sixth aspect, the vector comprises a nucleotide sequence encoding BDNF or NT-3.

In one embodiment, the vector comprises a nucleotide sequence encoding BDNF and NT-3, operably linked to non-coding sequences enabling transcription and/or translation. The vector may include nucleotide sequences such as promoter sequences enabling selective expression within cell types and may include sequences which are known to improve the translation efficiency of mRNA.

A seventh aspect provides a composition comprising the nucleic acid molecule of third to fifth aspect, or the vector of the sixth aspect, and a pharmaceutically acceptable carrier.

An eighth aspect provides a method of increasing BDNF and/or NT-3 production in a mammalian cell, comprising introducing into the cell the nucleic acid molecule of the third to fifth aspect, the vector of the sixth aspect, or the composition of the seventh aspect.

In one embodiment of the tenth to thirteenth aspect, the nucleic acid molecule is an RNA molecule, typically an mRNA molecule.

A ninth aspect provides a method of promoting neural growth and/or neural survival in mammalian tissue, comprising introducing into cells of the tissue the recombinant nucleic acid molecule of the third to fifth aspect, the vector of the sixth aspect, or the composition of the seventh aspect.

A tenth aspect provides a method of generating neural growth and/or neural survival in mammalian tissue, the method comprising:
(a) inserting two or more electrodes at a target region in the mammalian tissue;
(b) introducing to the tissue in the target region RNA comprising nucleotide sequence capable of expressing a protein interest in the one of more cells of the tissue;
(c) inducing via the two or more electrodes an electric potential difference in the tissue of the target region, the electric potential difference causing an electric field through the target region to thereby promote entry of the mRNA into the mammalian tissue,
   wherein the protein of interest is human BDNF protein and/or human NT-3 protein, and wherein the nucleotide sequence encoding the BDNF and/or NT-3 protein is codon optimised for translation of the protein in a mammalian cell at a level that is higher relative to the level of protein translation from a nucleotide sequence encoding the protein that is not codon optimised.

An eleventh aspect provides a kit comprising the nucleic acid molecule of the third to fifth aspects, the vector of the sixth aspect, or the composition of the seventh aspect.

A twelfth aspect provides a method of generating neural growth and/or neural survival in the cochlea of a human, comprising introducing into cells of the cochlea the recombinant nucleic acid molecule of the third to fifth aspect, the vector of the sixth aspect, or the composition of the seventh aspect.

A thirteenth aspect provides a method of generating neural growth and/or neural survival in the cochlea of a human, the method comprising:
(a) inserting a probe shaped to mimic a cochlear implant and comprising two or more electrodes at a target region proximate to cells of the cochlear;
(b) introducing to the target region RNA comprising nucleotide sequence capable of expressing a protein of interest in the one of more cells of the tissue;
(c) inducing via the two or more electrodes an electric potential difference in the target region, the electric potential difference causing an electric field through the target region to thereby promote entry of the RNA into cells of the cochlea,
   wherein the protein of interest is human BDNF protein and/or human NT-3 protein, and wherein the nucleotide sequence encoding the BDNF and/or NT-3 protein is codon optimised for translation of the protein in a mammalian cell at a level that is higher relative to the level of protein translation from a nucleotide sequence encoding the protein that is not codon optimised.

A fourteenth aspect provides a method of improving the hearing of a human in need thereof, comprising introducing into cells of the cochlea the nucleic acid molecule of the third to fifth aspect, the vector of the sixth aspect, or the composition of the seventh aspect.

A fifteenth aspect provides a method of improving the hearing of a human in need thereof, comprising:
(a) inserting a probe shaped to mimic a cochlear implant and comprising two or more electrodes at a target region proximate to cells of the cochlea;
(b) introducing to the target region RNA comprising nucleotide sequence capable of expressing a protein of interest in the one of more cells of the tissue;
(c) inducing via the two or more electrodes an electric potential difference in the target region, the electric potential difference causing an electric field through the target region to thereby promote entry of the RNA into cells of the cochlea,
   wherein the protein of interest is human BDNF protein and/or human NT-3 protein, wherein the nucleotide sequence encoding the BDNF and/or NT-3 protein is codon optimised for translation of the protein in a mammalian cell at a level that is higher relative to the level of protein translation from a nucleotide sequence encoding the protein that is not codon optimised.

### BRIEF DESCRIPTION OF THE FIGURES

FIG. 1 is a comparison of native and codon optimised coding sequence of human brain-derived neurotrophic factor (BDNF) DNA. The top sequence is the native human BDNF coding sequence identical to INSDC accession #M37762.1—Human brain-derived neurotrophic factor (BDNF) gene, complete cds. The underlying sequence is codon optimised version of the BDNF coding sequence. Codons substituted for individual amino acids are highlighted. Absence of an underlying star (*) corresponds to a base change specific to our therapeutic BDNF-encoding DNA sequence.

FIG. 2 is a comparison of native and codon optimised coding sequence of human brain-derived neurotrophic factor (BDNF) RNA. The underlying sequence is codon optimised version of the BDNF mRNA coding sequence. Codons substituted for individual amino acids are highlighted. Absence of an underlying star (*) corresponds to a base change specific to the BDNF encoding DNA sequence.

FIG. 3 is a comparison of native and codon optimised coding sequence of human neurotrophin-3 (NT3) DNA. The top sequence is the native human NT3 coding sequence identical to INSDC accession #M37763.1—Human neurotrophin-3 (NT-3) gene, complete cds. The bottom sequence is a codon optimised version of NT3. Absence of an underlying star (*) corresponds to a base change specific to our therapeutic DNA sequence.

FIG. 4 is a comparison of native and codon optimised coding sequence of human neurotrophin-3 (NT3) RNA. The underlying sequence is codon optimised version of the NT3 mRNA coding sequence. Codons substituted for individual amino acids are highlighted. Absence of an underlying star (*) corresponds to a base change specific to the NT3-encoding DNA sequence.

FIG. 5 is images showing translation of green fluorescent protein expression in mouse hindlimb tissue from plasmid DNA introduced using electrotransfer. High efficiency gene expression was achieved in vivo in the mouse hindlimb muscle following BaDGE® electrotransfer of a green fluorescent protein (GFP)—encoding plasmid DNA (2 μg/μl; 50 μl; CMVp-GFP in 10% sucrose carrier) using a multi-pulse protocol (10×100 μs pulses at 400 μs intervals, 50 mA/60 V) delivered via BaDGE® electrotransfer. 50 μm cryosections, 488 nm excitation confocal images 4 days after electrotransfer.

FIG. 6 shows single capacitive discharge electrotransfer modelled against conventional square wave pulse electrotransfer in HEK293 cells. Discharge time constants of 4 ms or longer produced more GFP reporter expression than a pulse train of 10×100 μs pulses (50 mA). FIG. 6 is a graph showing mCherry reporter fluorescence from coverslips of HEK293 cells (n=5 per treatment), where plasmid DNA encoding the reporter was placed on the cells and the BaDGE delivery probe used to electrotransfer the DNA into cells by single capacitive discharge electrotransfer or by conventional square wave pulse electrotransfer. Below the graph are images of the coverslips expressing mCherry after 3 days in tissue culture. Data shows equivalency between a reference 10×100 μs square wave constant current pulses (~50 mA per pulse-designated as “100Up 100 us square” and 1 single capacitive discharge with a 400 us time constant (“1UP 400 us decay constant”), a 1 millisecond time constant (e.g., “1 UP 1 ms decay constant”), a 4 ms time constant (“1UP 4 ms decay constant”) or a 10 ms time constant (“1 UP 10 ms decay constant”). Single capacitance discharges with longer time constants showed significantly improved gene electrotransfer efficiency.

FIG. 7A is a diagram showing the structure of plasmid reporter (luciferase) pMK175, which was delivered using single capacitive discharge gene electrotransfer into mouse hindlimbs. FIG. 7B is an image showing luciferase expression in mouse following electrotransfer of pMK175 by single capacitive discharge gene electrotransfer or by conventional square wave pulse train delivery into mouse hindlimbs. FIG. 7C is a graph showing expression in mouse hindlimb of luciferase from pMK175 over time comparing conventional square wave pulse train delivery with single capacitive discharge delivery; readout is repeated quantitative in vivo bioluminescence recording from the same mouse.

FIG. 8 is graphs showing a comparison of in vivo luciferase expression from naked mRNA and DNA, each encoding luciferase protein, which have been introduced into hindlimb muscle using electrotransfer via a BEPv1 electrotransfer probe and Single Capacitive Discharge control unit 2 with discrete capacitor charging. Luciferase-encoding plasmid DNA (CAGp-Luc; 0.5 μg/μl (30 μl)) was delivered by BaDGE® electrotransfer into one hindlimb and luc-mRNA (0.5 μg/μl; 30 μl; Trilink cat. L-7202) was delivered into the opposite hindlimb of C57BL/6J adult mice using single capacitive discharge (2.2 μF at 120 V) using a BaDGE® BEPv1 electrotransfer probe and BaDGE® Single Capacitive Discharge control unit 2 (BSCDcu2). Luciferase reporter expression with repeated measures readout across 2 weeks (IVIS Spectrum CT bioluminescence imaging platform under isoflurane anaesthesia). Graphs show higher luciferase protein-mediated bioluminescence following mRNA delivery over pDNA from the first measurement at 10 hours, out to 14 days (data shown for 10 hours, 1, 3, 7 & 14 days). FIG. 8A is a graph showing longitudinal data across individual legs. FIG. 8B is a graph showing an average of the data (mean±s.e.m.) with repeated measures using two way ANOVA comparing mRNA and DNA expression. P=0.019; n=5 per group.

FIG. 9A shows image of BaDGE® BSCDp1 delivery probe for use with a docking station (part ref. BSCDds1) as described in WO 2021/72507.

FIG. 9B is in vivo imaging of luciferase (Luc) bioluminescence in isoflurane anaesthetised mice 1-3 days after BaDGE® single capacitive discharge (SCD) electrotransfer delivery to mouse hindlimb muscle of mRNA (Luc mRNA (30 μl @ 0.5 μg/μl; Trilink cat. L-7202) and DNA encoding luciferase (CAGp.luc) vs DNA (30 μl @ 2 μg/μl plasmid MK175—Lux)). This utilized the BaDGE® single capacitive discharge delivery probe and docking station (2.2 μF, 200 V) showing enhanced expression from mRNA over DNA from 1 and 3 days (BALB/c mouse strain). Signal is pseudocoloured readout of bioluminescence intensity following intraperitoneal injection of luciferin substrate, measured using a IVIS Spectrum CT imaging platform.

FIG. 10 is an image confirming expression of luciferase (Luc) protein in hindlimb muscle of BALB/c mouse hindlimb muscle using immunohistochemistry. Fixed tissue was cryosectioned and immunolabelled using an anti-luciferase antibody (50 μm cryosection) 24 hrs after BaDGE® electrotransfer of Luc-encoding mRNA (Trilink cat. L-7202; 0.5 μg/μl; 2.2 μF, 200 V single capacitive discharge). Anti-Lux antibody (Thermo #35-6700; mouse monoclonal; 25 μg/ml (1:20)); detected using an Alexa488 goat antimouse secondary. Nuclei labelled with DAPI.

FIG. 11 is a graph showing Luc-mRNA expression onset in adult BALB/cJ mouse hindlimb in vivo (luciferase bioluminescence) with single measurements at 3 h, 12 h time; 24 h, 48 h and 96 h following luc-mRNA (Trilink L-7202; 0.5 mg/ml in BaDGE®-carrier© solution) delivery using the BaDGE® electrotransfer. Bioluminescence (total photon flux) was measured (IVIS Spectrum CT) at 3 h (n=4; P=0.0373 one-sample t-test), 12 h (n=6), 24 h (n=4; P=0.002 compared with 12 h, ANOVA Holm-Sidak multiple pairwise comparisons), 48 h (n=4; P<0.001 compared with 3 h & 12 h) and 96 h (n=4; P=0.023 compared with 48 h). Box plots show 25% and 75% range, median and mean (dashed lines), with 95% confidence limits and individual data overlay. BaDGE® single capacitive discharge delivery probe (part ref. BSCDp1) and docking station (part ref. BSCDds1; 2.2 μF, 200 V).

FIG. 12 is a graph showing Luc-mRNA expression onset and decay compared with DNA expression in adult BALB/cJ mouse hindlimb in vivo (luciferase bioluminescence) following BaDGE® electrotransfer. A control treatment groups with no electrotransfer was included for mRNA and DNA delivery. Temporal expression profiles of the luciferase reporter delivered either as plasmid DNA in BaDGE®-carrier® solution (2 μg/μl; CAG promoter) or as mRNA (Trilink L-7202; 0.5 μg/μl in BaDGE®-carrier® solution) using the BaDGE® electrotransfer. Bioluminescence (total photon flux was measured periodically at 1, 3, 7, 14, 28, 56, 80 days following electrotransfer. Controls were no electrotransfer mRNA and DNA groups. Two way ANOVA (RM) identified significant differences between BaDGE® expression for Luc-mRNA vs Luc-DNA; note inflection at day 7 from mRNA>DNA to mRNA<DNA (1 day P=0.003; 3 days P=0.014; 7 days P=0.551; 14 days P=0.01; 28 days P=0.062; 56 days P=0.088; 80 days P=0.024). ‘No-BaDGE’ (injected mRNA/DNA without electrotransfer pulse) showed minimal signal (P<0.001 compared with BaDGE® electrotransfer, three way ANOVA). BaDGE® single capacitive discharge delivery probe (part ref. BSCDp1) and docking station (part ref. BSCDds1; 2.2 μF, 200 V). Adult BALB/cJ mice. Each mouse received DNA to one hindlimb and mRNA to the other (randomized).

FIG. 13 is an image demonstrating auditory nerve regeneration in vivo following BaDGE® electrotransfer-based delivery of the OHBNEC-CMVp-BDNF-IRES-NT3 plasmid DNA to the perilymphatic compartment of the cochlea. This image shows neuron-specific TUBB3 ((3Tubulin) immunofluorescence labelling of the basal region spiral ganglion neurons (SGN) in a deafened guinea pig cochlea four weeks post deafening and two weeks post OHBNEC BaDGE® electrotransfer. Arrows indicate ectopic branching of the regenerated SGN peripheral neurite processes beyond the location where they would normally terminate to synapse with the sensory hair cells. The experimental protocol was approved by the UNSW Animal Care and Ethics Committee.

FIG. 14 is an image of the BaDGE® DNA delivery probe version 1 used to electrotransfer RNA and DNA in some embodiments described herein, and as described in WO 2020/118383. The electrode comprises twin Pt/Ir tube electrodes (2 mm length×400 μm outside diameter on an insulated 34 G needle (overall equivalent to a 27 G needle). Twin insulated wires are terminated as a gold-plated mini-plug which connects to the leads of the BaDGE® Control Unit (BCU1) for constant current square wave pulse delivery.

FIG. 15 is a graph of BDNF and NT3 protein levels as determined by ELISA in guinea pig perilymph following electrotransfer of BDNF-T2A-NT3 mRNA using Cochlear Implant BaDGE® as compared to contralateral untreated control up to 1 week after treatment (n=4). The data shows a significant effect of the treatment (P<0.001). Two-way ANOVA. Box plots reflect 25% and 75% quartiles, with data overlay. Dashed lines show mean values; solid lines show the median and error bars outline the 95th percentile confidence intervals.

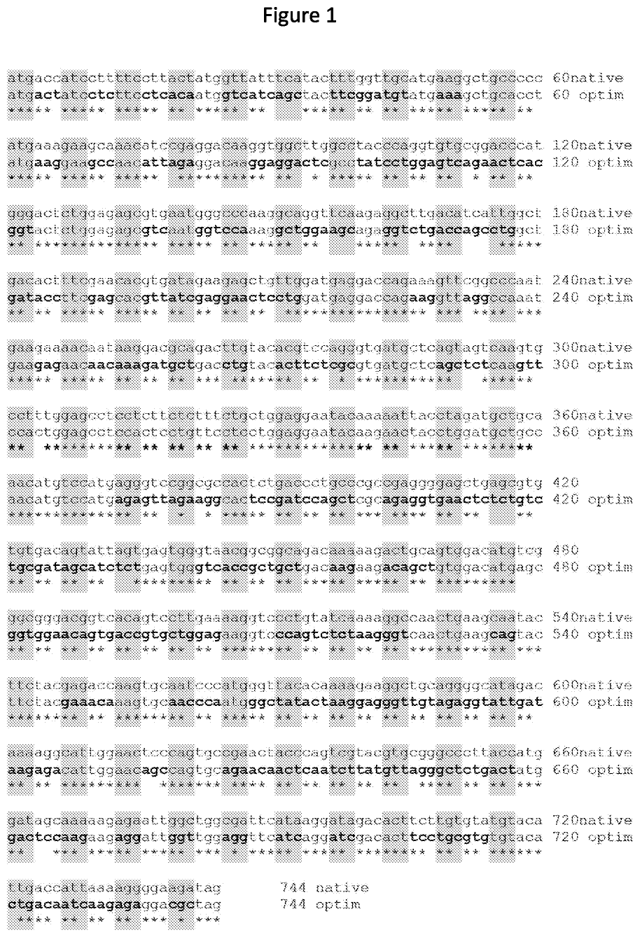

### DETAILED DESCRIPTION

The present disclosure relates in one embodiment to methods of promoting rapid production of a recombinant protein of interest in cells of a tissue in vivo. The method comprises transferring mRNA encoding a protein of interest to be expressed into cells of a tissue by applying an electric field to the cells of the tissue sufficient to promote entry of the mRNA into the cells. Such a transfer of a nucleic acid molecule into cells of a tissue is referred to herein as electrotransfer (or electroporation) of the nucleic acid molecule into the cells.

The inventors have found that by introducing mRNA, typically naked mRNA, encoding a protein of interest into cells of a tissue using electrotransfer, translation of the protein encoded by the mRNA is detectable at high levels within 1-3 days of delivery. Such rapid expression of the protein is particularly advantageous in situations where delay can be detrimental, such as in promoting neural growth/survival in certain circumstances, such as following neural injury or neural insult.

Accordingly, one aspect provides a method of expressing a protein of interest in one or more cells of a tissue in a target region, the method comprising:
(a) introducing to the tissue in the target region RNA, typically mRNA, capable of expressing the protein of interest in the one or more cells; and
(b) inducing via two or more electrodes proximate the target region an electric potential difference in the tissue of the target region, the electric potential difference causing electric fields through the target region, to thereby promote entry of the mRNA into the one or more cells of the tissue.

As used herein, the expression “expressing a protein of interest in one or more cells of a tissue” refers to producing a protein of interest in one or more cells of tissue, typically producing a recombinant protein of interest in one or more cells of tissue.

The one or more cells of tissue, may be one or more cells of a single tissue, or one or more cells of a plurality of tissues.

Expression of a protein from DNA using the same delivery method (electrotransfer) results in slower expression, and lower levels of protein expression. As described in the Examples, when luciferase protein was expressed from DNA in vivo following electrotransfer, maximum expression was detected at 14 days as compared to maximum expression from mRNA encoding luciferase being detected at 3 days. Moreover, the amount of protein expression detected was significantly greater from mRNA delivery that from DNA delivery.

Further, delivery of mRNA using viral delivery, liposomes or nanoparticles usually takes more time for the mRNA to express the protein of interest. In contrast, delivery of mRNA by electrotransfer as described herein, results in maximal protein expression within as little as 1-3 days.

As used herein, an “RNA capable of expressing the protein of interest” is an RNA molecule comprising nucleotide sequence which encodes the protein of interest, and includes the necessary ribosome binding sites and signal sequences to permit translation of the coding sequence when in the one or more cells of the tissue.

Typically, the RNA is mRNA.

Typically, the mRNA is naked RNA. By using naked mRNA, little preparation of the mRNA is required other than its production. This therefore avoids the necessity for packaging the mRNA in liposomal or other delivery vehicles, and thus avoids the complexity and expense of the manufacturing process associated with packaging of mRNA into liposomes or other such delivery vehicles.

In one embodiment, the mRNA molecule comprises modified nucleotides. mRNA comprising modified nucleotides may comprise, for example, a modified sugar moiety, a modified internucleoside linkage, a modified nucleoside, a modified nucleotide, and/or combinations thereof. The modified mRNA may exhibit one or more of the following properties: are not immune stimulatory; are nuclease resistant; have improved cell uptake compared to unmodified donor polynucleotides; and/or are not toxic to cells or mammals

Specific examples of modified oligonucleotides include those comprising modified backbones (i.e. modified internucleoside linkage), for example, phosphorothioates, phosphotriesters, methyl phosphonates, short chain alkyl or cycloalkyl intersugar linkages or short chain heteroatomic or heterocyclic intersugar linkages. In some embodiments, mRNA may have phosphorothioate backbones; heteroatom backbones, such as methylene(methylimino) or MMI backbones; amide backbones; morpholino backbones; or peptide nucleic acid (PNA) backbones. Phosphorus-containing modified linkages include, for example, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

In some embodiments, the mRNA molecule encodes more than one protein. In some embodiments, the mRNA may encode two or more proteins. Typically, when the mRNA comprises nucleotide sequence encoding two or more proteins, the region in between the proteins typically comprises a Kozak sequence and an internal ribosome engagement site (IRES).

In one embodiment, the mRNA comprises a nucleotide sequence encoding human BDNF protein, wherein the nucleotide sequence is codon optimised to express BDNF protein in a mammalian cell at a level that is higher relative to the level of BDNF protein expressed from a nucleotide sequence encoding human BDNF protein that is not codon optimised.

In one embodiment, the amino acid sequence of the human BDNF protein is:

(SEQ ID NO: 1)
MTILFLTMVISYFGCMKAAPMKEANIRGQGGLAYPGVRTHGTLESVNGPK
AGSRGLTSLADTFEHVIEELLDEDQKVRPNEENNKDADLYTSRVMLSSQV
PLEPPLLFLLEEYKNYLDAANMSMRVRRHSDPARRGELSVCDSISEWVTA
ADKKTAVDMSGGTVTVLEKVPVSKGQLKQYFYETKCNPMGYTKEGCRGID
KRHWNSQCRTTQSYVRALTMDSKKRIGWRFIRIDTSCVCTLTIKRGR

In one embodiment, the nucleotide sequence of the mRNA encoding the human BDNF protein comprises the nucleotides sequence:

(SEQ ID NO: 2)
augacuauccucuuccucacaauggucaucaguacuucggauguaugaaa
gcugcaccuaugaaggaagccaacauuagaggacaaggaggacucgccua
uccuggagucagaacucacgguacucuggagagcgucaaugguccaaagg
cuggaagcagaggucugaccagccuggcugauaccuucgagcacguuauc
gaggaacuccuggaugaggaccagaagguuaggccaaaugaagagaacaa
caaagaugcugaccuguacacuucucgcgugaugcucagcucucaaguuc
cacuggagccuccacuccuguuccuccuggaggaauacaagaacuaccug
gaugcugccaacauguccaugagaguuagaaggcacuccgauccagcucg
cagaggugaacucucugucugcgauagcaucucugagugggucaccgcug
cugacaagaagacagcuguggacaugagcgguggaacagugaccgugcug
gagaaggucccagucucuaagggucaacugaagcaguacuucuacgaaac
aaagugcaacccaaugggcuauacuaaggaggguuguagagguauugaua
agagacauuggaacagccagugcagaacaacucaaucuuauguuagggcu
cugacuauggacuccaagaagaggauugguuggagguucaucaggaucga
cacuuccugcguguguacacugacaaucaagagaggacgcuag

In one embodiment, the mRNA further comprises a nucleotide sequence encoding human NT-3 protein.

In one embodiment, the amino acid sequence of the human NT-3 protein is:

(SEQ ID NO: 3)
MSILFYVIFLAYLRGIQGNNMDQRSLPEDSLNSLIIKLIQADILKNKLSK
QMVDVKENYQSTLPKAEAPREPERGGPAKSAFQPVIAMDTELLRQQRRYN
SPRVLLSDSTPLEPPPLYLMEDYVGSPVVANRTSRRKRYAEHKSHRGEYS
VCDSESLWVTDKSSAIDIRGHQVTVLGEIKTGNSPVKQYFYETRCKEARP
VKNGCRGIDDKHWNSQCKTSQTYVRALTSENNKLVGWRWIRIDTSCVCAL
SRKIGRT

In one embodiment, nucleotide sequence encoding the human NT-3 protein is codon optimised to express the human NT-3 protein in the mammalian cell at a level that is higher relative to the level of NT-3 protein expressed from a nucleotide sequence encoding human NT-3 protein that is not codon optimised.

In one embodiment, the codon optimised nucleotide sequence encoding human NT-3 comprises the nucleotide sequence:

(SEQ ID NO: 4)
augucuauccuguucuaugugaucuuccucgcauaccugagaggaaucca
aggcaacaacauggaucaacgcagccugccagaagacagccugaacucuc
ugaucaucaagcucauccaagcagacauucugaagaacaagcuguccaaa
cagaugguggaugucaaagagaacuaucaguccacucugcccaaagcaga
ggcucccagggagccagaaagagguggaccugccaagucugcuuuccaac
cagugauugcaauggacacugagcugcucaggcagcagaggagguacaau
ucucccaggguucugcucucugauuccacuccucuggaaccaccaccucu
cuaccugauggaggauuacguggguucuccagugguggcaaacagaacaa
gcagacgcaagagguaugcagaacacaaaucccacagaggugaguauagc
gugugcgacucugaaucucugugggugacugacaaauccucugcuauuga
cauuagaggacaucaggugacagugcucggagagaucaagacuggcaaua
gcccagucaaacaauacuucuacgaaacucgcuguaaagaggcaaggcca
guuaagaacgguuguagagguaucgacgacaaacacuggaauagccagug
uaagacuucucaaaccuacguuagagcccucacaucugagaacaacaagc
ucgucggauggagguggaucagaauugauaccuccugcgucugugcacug
agcaggaagauuggaagaaccuaa

In one embodiment, the mRNA comprises nucleotide sequence encoding BDNF and NT-3.

In one embodiment, the mRNA comprises nucleotide sequence encoding BDNF and NT-3, and comprises nucleotide sequence encoding a T2A element between the BDNF and NT-3 coding sequence, wherein the T2A element is autocleaved during protein translation.

A second aspect provides a method of expressing a protein of interest in one or more cells of a tissue, the method comprising:
(a) inserting two or more electrodes at a target region comprising the one or more cells of the tissue;
(b) introducing to the target region mRNA capable of expressing the protein of interest;
(c) inducing via the two or more electrodes an electric potential difference in the target region, the electric potential difference causing an electric field through the target region sufficient to promote entry of the RNA into the one or more cells of the tissue,
   wherein the protein of interest is human BDNF protein and/or human NT-3 protein, and wherein the nucleotide sequence encoding the BDNF and/or NT-3 protein is codon optimised for translation of the protein in a mammalian cell at a level that is higher relative to the level of protein translation from a nucleotide sequence encoding the protein that is not codon optimised.

In various embodiments, the protein of interest is expressed within:
4 days of inducing the electric field;
3 days of inducing the electric field;
2 days of inducing the electric field;
1 day of inducing the electric field;
12 hours of inducing the electric field;
6 hours of inducing the electric field;
4 hours of inducing the electric field; or
2 hours of inducing the electric field.

In one embodiment, the electric potential difference causes electric fields in the range of from 50 μV/μm to 25,000 μV/μm, 100 μV/μm to 25,000 μV/μm, 200 μV/μm to 25,000 μV/μm, 500 μV/μm to 25,000 μV/μm, 1,000 μV/μm to 25,000 μV/μm, 1,500 μV/μm to 25,000 μV/μm, 2,000 μV/μm to 25,000 μV/μm, 3,000 μV/μm to 25,000 μV/μm, 4,000 μV/μm to 25,000 μV/μm, 5,000 μV/μm to 25,000 μV/μm, 6,000 μV/μm to 25,000 μV/μm, 7,000 μV/μm to 25,000 μV/μm, 8,000 μV/μm to 25,000 μV/μm, 9,000 μV/μm to 25,000 μV/μm, 10,000 μV/μm to 25,000 μV/μm, 1,000 μV/μm to 24,000 μV/μm, 1,000 μV/μm to 23,000 μV/μm, 1,000 μV/μm to 22,000 μV/μm, 1,000 μV/μm to 21,000 μV/μm, 1,000 μV/μm to 20,000 μV/μm, 1,000 μV/μm to 19,000 μV/μm, 1,000 μV/μm to 18,000 μV/μm, v, 1,000 μV/μm to 17,000 μV/μm, 1,000 μV/μm to 16,000 μV/μm, 1,000 μV/μm to 15,000 μV/μm, 1,000 μV/μm to 14,000 μV/μm, 1,000 μV/μm to 13,000 μV/μm, 1,000 μV/μm to 12, 1,000 μV/μm to 11,000 μV/μm, 000 μV/μm, 1,000 μV/μm to 10,000 μV/μm, 2,000 μV/μm to 24,000 μV/μm, 2,000 μV/μm to 23,000 μV/μm, 2,000 μV/μm to 22,000 μV/μm, 2,000 μV/μm to 21,000 μV/μm, 2,000 μV/μm to 20,000 μV/μm, 2,000 μV/μm to 19,000 μV/μm, 2,000 μV/μm to 18,000 μV/μm, 2,000 μV/μm to 25,000 μV/μm, 2,000 μV/μm to 17,000 μV/μm,.

The electrodes may be any configuration that is suitable to inserting into the tissue. Conventional electroporation places electrodes on either side of target tissue to apply electric charge to the target region. The inventors have developed an alternative method for targeted electrotransfer using an in-line linear array of electrodes to generate shaped electric fields adjacent the electrode array in a target region. The electrodes are arranged in line, physically contiguous with the probe, such that the probe structure has alternating sections of electrode and insulating material. With this structure, then the probe is inserted into tissue, the tissue is physically adjacent the electrodes rather than between electrodes. Electric fields generated in the adjacent tissue are focussed by the gaps between the electrodes focus electric fields generated by passing current between electrodes driven as anodes and electrodes driven as cathodes. The electric field strength will vary along the length of the probe, with the electric field induced in the biological tissue having highest electric field strength extending orthogonal to the point between the anode and cathode. This method has an advantage of enabling a single probe (or needle array) to be used. Suitable electrodes and further explanation of the method and system for electrotransfer are described in WO2016/205895, WO2014/201511, WO2011/006204, WO2020/118383 and WO2021/072507.

In one embodiment, the electrodes are configured as described in WO 2020/118383.

In one embodiment, the electrodes are configured as described in WO 2021/072507.

In one embodiment, the two or more electrodes are relatively elongate along the direction of contiguity, wherein the electrode circumference is less than the electrode length. This configuration induces electric field gradients concentrated at the gap between the electrodes driven as anodes and cathodes. The length of the electrodes and gap length can be designed to control the generated electric field gradients.

In some embodiments, the two or more electrodes comprises an anode or anode array and a cathode or cathode array, and wherein the electric field is created by driving the anode or anode array and the cathode or cathode array in said target region for sufficient time to allow at least some of the RNA molecules to enter the one or more cells.

In some embodiments, the anode or anode array and the cathode or cathode array are separated by between about 10 μm and about 5 mm.

In some embodiments, the anode or anode array and the cathode or cathode array are provided on a single probe to be introduced into the target region.

In one embodiment, the anode or anode array and the cathode or cathode array are provided on a probe configured to mimic the shape of a cochlear implant electrode array.

In some embodiments, each of the arrays independently comprises from 2 to 8 electrodes. In various embodiments, each of the arrays comprises 2, 3, 4, 5, 6, 7, or 8, electrodes.

In some embodiments, each of the arrays comprises 4 electrodes.

In one embodiment, the electrode is shaped to model a cochlear implant prosthesis to be inserted into the cochlea for performing electroporation and subsequently removed, the electrode being electrically conductive at its surface along one or more portions of the entire length of the electrode, and electrically non-conductive at its surface along other portions of its length.

The electric field may be induced using any electroporation system that causes the RNA to enter the cell. Suitable electroporation systems include those described in, for example, WO2016/205895, WO2014/201511, WO2011/006204, WO2020/118383 and WO2021/072507.

In one embodiment, the electric field is induced using an electroporation system comprising a controller configured to allow selective control of a pulse generator to control the sequence of pulses delivered via the electrode, and wherein the controller is further configured to control electrical pulse parameters for the sequence of pulses based on carrier solution characteristics.

In one embodiment, the total electric charge delivered during the electroporation is less than 5000 μCoulombs, typically less than 500 μCoulombs, more typically less than 1 μCoulombs.

In one embodiment, the electric charge is delivered through less than 100 electric pulses, optionally through 10 or fewer pulses, 5 or fewer pulses, 4 pulses, 3 pulses, 2 pulses or 1 pulse.

In various embodiments, each pulse is from about 100 us to about 500 ms in duration.

In one embodiment, the electric potential difference is induced using an electroporation system comprising:
an array of two or more physically contiguous electrodes configured to be inserted into the tissue;
   a pulse generator electrically connected to the electrodes of the array and configured to apply one or more electrical pulses to selectively drive the two or more electrodes as one or more anodes and one or more cathodes to generate an electric field in tissue adjacent the array, wherein the electric field is shaped, to provide controlled contours of voltage gradients within the electric field, based on the physical configuration of the electrodes, selection of electrodes and anodes and cathodes, and applied electrical pulse parameters; and
a controller configured to control the pulse generator, the controller being configured to control the pulse generator to apply a first sequence of one or more unipolar pulses using a first configuration of electrodes driven as anodes and cathodes to provide a first shaped electric field, and a second sequence of one or more unipolar pulses using a second configuration of electrodes driven as anodes and cathodes to provide a second shaped electric field.

In one embodiment, the electric potential difference is induced using an electrotransfer system comprising:
at least one probe, each probe comprising:
   a probe body;
   a needle electrode array extending from the probe body configured as a needle to be inserted into tissue to be treated; and
   a capacitive discharge circuit connected to the needle electrode array comprising capacitive charge storage configured to store a quantum of charge, and a switch actuatable to cause discharge of the stored quantum of charge through the needle electrode array,
   the needle electrode array incorporating at least two electrodes, each electrode having a surface area substantially circumferential to the needle and exposed to directly contact tissue into which the needle electrode array is inserted, with an insulating section between neighbouring electrodes to form a contiguous linear array structure, the exposed surface area of each electrode being at a different distance from the needle electrode array tip, each electrode connected to the capacitive discharge circuit for driving as an anode or cathode during discharge of the quantum of charge, and wherein electrode length and length of the insulating section between neighbouring electrodes are configured to produce a target electric field shape in tissue adjacent to the array during discharge of the quantum of charge via the array. Typically fluidics channels within the electrotransfer probe would provide delivery of the therapeutic DNA/RNA molecules in the carrier to the centre of the pulsed electric field established by the electrode configuration.

In one embodiment, the electrotransfer system comprises a charging station having a DC power supply module, output terminals connectable to a probe to form electric contact to the capacitive discharge circuit, and charging control circuitry to control charging of the capacitive discharge circuit of the connected probe.

In one embodiment, the needle electrode comprises:
a first needle which also acts as a first electrode;
a first concentric insulator sheathing the first needle to a predetermined first distance (L1) from the needle tip,
a concentric second electrode sheathing the first concentric insulator to a distance (L2) from a tipward end of the first concentric insulator, and
a second concentric insulator sheathing the second electrode to a distance (L3) from a tipward end of the second electrode, wherein the first needle and second concentric electrode are formed of conductive material and are electrically connected to positive and negative terminals of the capacitive discharge circuit.

In one embodiment, the exposed length L1 of the first needle operates as a first electrode of a linear array, and the exposed length L3 of the second concentric electrode acts as the second electrode of a linear array.

In one embodiment, the respective lengths of the first and second electrodes, and exposed length of the first insulating portion therebetween determines the pattern of electric field gradients generated adjacent the needle array when an electric pulse is applied to drive one electrode as an anode and the other electrode as a cathode.

The present disclosure also relates to nucleic acids comprising nucleic acid sequences encoding BDNF and/or NT-3 protein which have been codon optimised to achieve production of the BDNF and NT-3 proteins at higher levels in mammalian cells than the naturally occurring BDNF and/or NT-3 genes.

Brain-derived neurotrophic factor (BDNF) and neurotrophin-3 (NT-3) are neurotrophins which play an important role in neuronal survival and growth, and participate in neuronal plasticity. BDNF binds to its high affinity receptor TrkB (tyrosine kinase B) and activates signal transduction cascades (IRS1/2, PI3K, Akt), crucial for CREB and CREB Binding Protein production, that encode proteins involved in B cell survival. BDNF also binds the LNGFR receptor (low-affinity nerve growth factor receptor, also known as p75). NT-3 binds the TrkC receptor with the greatest affinity and TrkB and LNGFR with lesser affinity.

The inventors have optimised the codons of the coding sequence for the naturally occurring BDNF and NT-3 genes such that the coding sequence comprising the optimised codons results in production of the encoded protein at higher levels in mammalian cells than the nucleotide coding sequence of the naturally occurring genes. Introduction of the optimised BDNF and NT-3 coding sequences into mammalian cells results in production and secretion of the BDNF and NT-3 proteins at levels higher than would be observed in cells into which the optimised coding sequences were not introduced. The recombinant nucleic acids described herein can therefore be used to express and secrete relatively high levels of BDNF and/or NT-3 to thereby promote neuronal growth and/or support maintenance of neurons in tissue wherein this may not have previously been possible. In this regard, expression and secretion of the BDNF and/or NT-3 protein from cells comprising the recombinant nucleic acid molecules described herein can promote growth of nerve fibers and provide tropic action capable of steering outgrowth of the nerve fibres towards the cells secreting these recombinant nucleic acid-derived proteins.

In some embodiments, the recombinant nucleic acid is DNA.

In some embodiments, the recombinant nucleic acid is RNA. Typically, the RNA is mRNA.

In one embodiment, the nucleic acid is DNA and the optimised nucleotide sequence encoding BDNF comprises the following nucleotide sequence:

(SEQ ID NO: 5)
atgactatcctcttcctcacaatggtcatcagctacttcggatgtatgaa
agctgcacctatgaaggaagccaacattagaggacaaggaggactcgcct
atcctggagtcagaactcacggtactctggagagcgtcaatggtccaaag
gctggaagcagaggtctgaccagcctggctgataccttcgagcacgttat
cgaggaactcctggatgaggaccagaaggttaggccaaatgaagagaaca
acaaagatgctgacctgtacacttctcgcgtgatgctcagctctcaagtt
ccactggagcctccactcctgttcctcctggaggaatacaagaactacct
ggatgctgccaacatgtccatgagagttagaaggcactccgatccagctc
gcagaggtgaactctctgtctgcgatagcatctctgagtgggtcaccgct
gctgacaagaagacagctgtggacatgagcggtggaacagtgaccgtgct
ggagaaggtcccagtctctaagggtcaactgaagcagtacttctacgaaa
caaagtgcaacccaatgggctatactaaggagggttgtagaggtattgat
aagagacattggaacagccagtgcagaacaactcaatcttatgttagggc
tctgactatggactccaagaagaggattggttggaggttcatcaggatcg
acacttcctgcgtgtgtacactgacaatcaagagaggacgc.

In one embodiment, the nucleic acid is RNA and the optimised nucleotide sequence encoding BDNF comprises the nucleotide sequence of SEQ ID NO: 2.

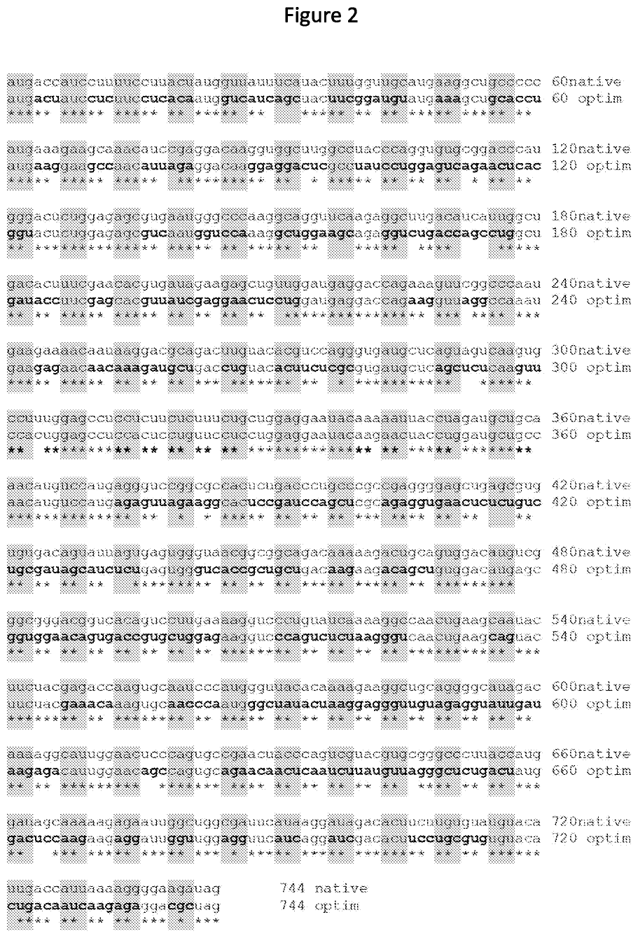

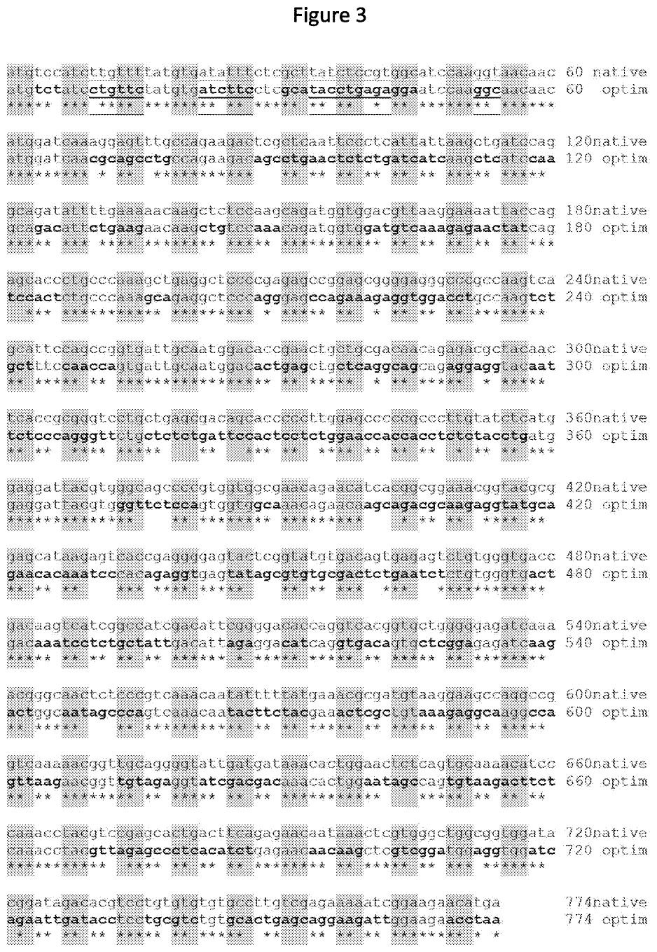

Depending upon the juxtaposition of encoded amino acids, codon optimization selected alternative codons for a particular amino acid to improve translation. These substitutions are evident in the optimised (optim) sequences for DNA and RNA encoding the human BDNF protein described herein. For example, at the first codon for threonine in the BDNF—encoding DNA sequence (position 4-6), the native codon was ‘acc’ and this was substituted for the alternative codon ‘act’ (also at position 115-117); whereas ‘act’ was native for encoding threonine at position 19-21, where-in the recombinant BDNF utilised ‘aca’. Note that ‘acg’ (not used) and ‘aca’ (used for six substitutions) are additional alternative codons for threonine. All candidate codons encoding the different amino acids within the BDNF protein were reviewed in this manner and modified as indicated in FIG. 2 (* indicates no nucleotide change).

In one embodiment, the nucleic acid is DNA and the optimised nucleotide sequence encoding NT-3 comprises the following nucleotide sequence:

(SEQ ID NO: 6)
atgtctatcctgttctatgtgatcttcctcgcatacctgagaggaatcca
aggcaacaacatggatcaacgcagcctgccagaagacagcctgaactctc
tgatcatcaagctcatccaagcagacattctgaagaacaagctgtccaaa
cagatggtggatgtcaaagagaactatcagtccactctgcccaaagcaga
ggctcccagggagccagaaagaggtggacctgccaagtctgctttccaac
cagtgattgcaatggacactgagctgctcaggcagcagaggaggtacaat
tctcccagggttctgctctctgattccactcctctggaaccaccacctct
ctacctgatggaggattacgtgggttctccagtggtggcaaacagaacaa
gcagacgcaagaggtatgcagaacacaaatcccacagaggtgagtatagc
gtgtgcgactctgaatctctgtgggtgactgacaaatcctctgctattga
cattagaggacatcaggtgacagtgctcggagagatcaagactggcaata
gcccagtcaaacaatacttctacgaaactcgctgtaaagaggcaaggcca
gttaagaacggttgtagaggtatcgacgacaaacactggaatagccagtg
taagacttctcaaacctacgttagagccctcacatctgagaacaacaagc
tcgtcggatggaggtggatcagaattgatacctcctgcgtctgtgcactg
agcaggaagattggaagaacc.

In one embodiment, the nucleic acid is RNA and the optimised nucleotide sequence encoding NT-3 comprises the nucleotide sequence of SEQ ID NO: 4.

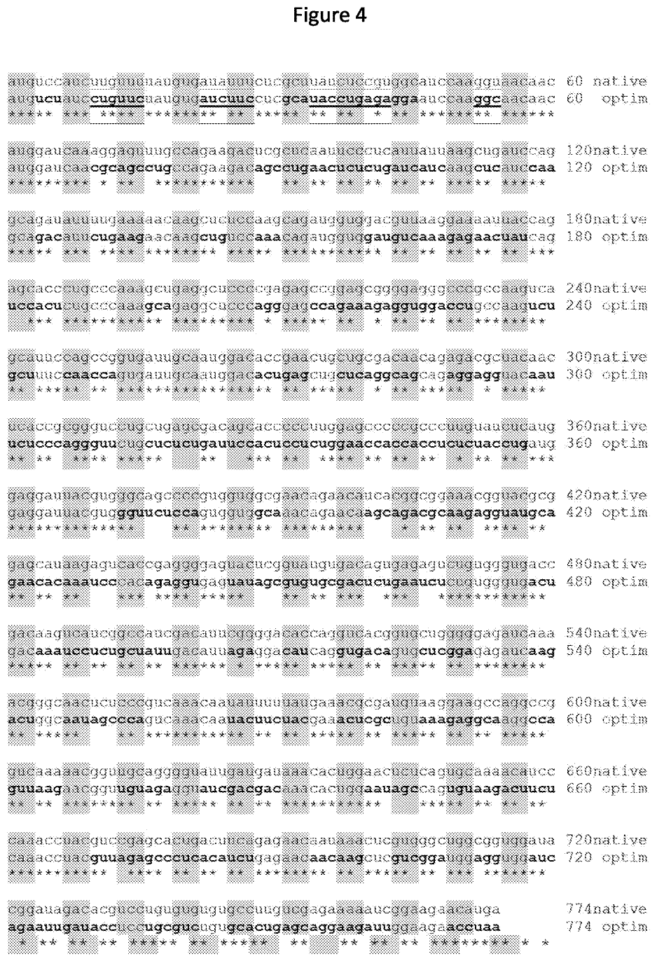

Depending upon the juxtaposition of encoded amino acids, codon optimization selected alternative codons for a particular amino acid to improve translation. These substitutions are evident in the above optimised (optim) sequences for DNA and RNA encoding the human NT-3 protein, as for the optimisation of the DNA and RNA sequences encoding the BDNF protein described above. All candidate codons encoding the different amino acids within the NT-3 protein were reviewed in this manner and modified as indicated in FIG. 4 (* indicates no nucleotide change).

The RNA and DNA nucleic acid sequence typically include regulatory sequences. A “regulatory sequence” is a nucleotide sequence located upstream (5′ non-coding sequences), within, or downstream (3′ non-coding sequences) of a coding sequence, and which influences the transcription, RNA processing or stability, or translation of the associated coding sequence. “Coding sequence” refers to a DNA or RNA sequence that codes for a specific amino acid sequence. Regulatory sequences are known in the art and may include, for example, transcriptional regulatory sequences such as promoters, enhancers, translation leader sequences, introns, and polyadenylation signal sequences. For DNA, the coding sequence is typically operably linked to a promoter. A promoter is a DNA region capable under certain conditions of binding RNA polymerase and initiating transcription of a coding sequence usually located downstream (in the 3′ direction) from the promoter.

In embodiments in which the nucleic acid is DNA, the nucleotide sequence encoding BDNF is typically operable linked to a promoter for expression of the BDNF protein in a mammalian cell.

In embodiments in which the nucleic acid is DNA, the nucleotide sequence encoding NT-3 is typically operable linked to a promoter for expression of the NT-3 protein in a mammalian cell.

In embodiments in which the nucleic acid is DNA, the nucleotide sequence encoding BDNF and NT-3 is typically operable linked to a promoter for expression of the BDNF and BT-3 proteins in a mammalian cell.

A nucleic acid encoding a protein (coding sequence) is operably linked to a regulatory sequence when it is arranged relative to the regulatory sequence to permit expression of the protein in a cell. For instance, a promoter is operatively linked to a coding region if the promoter helps initiate transcription of the coding sequence.

As used herein, “expression” of a nucleotide sequence refers to the transcription and/or translation of a nucleotide sequence comprising a coding sequence to produce the polypeptide encoded by the coding sequence.

In one embodiment, there is provided a nucleic acid molecule comprising a nucleotide sequence encoding human BDNF protein operably linked to a promoter for expression of the BDNF protein in a mammalian cell, wherein the nucleotide sequence is codon optimised to express BDNF protein in a mammalian cell at a level that is higher relative to the level of BDNF protein expressed from a nucleotide sequence encoding human BDNF protein that is not codon optimised.

In one embodiment, there is provided a nucleic acid molecule comprising a nucleotide sequence encoding human NT-3 protein operably linked to a promoter for expression of the NT-3 protein in a mammalian cell, wherein the nucleotide sequence is codon optimised to express NT-3 protein in a mammalian cell at a level that is higher relative to the level of NT-3 protein expressed from a nucleotide sequence encoding human NT-3 protein that is not codon optimised.

In one embodiment, the nucleic acid comprises a nucleotide sequence encoding human BDNF and human NT-3, each nucleotide sequence being operably linked to a promoter for expression of the BDNF and BT-3 protein in a mammalian cell, wherein the nucleotide sequence encoding the BDNF and NT-3 proteins is codon optimised to express each protein in a mammalian cell at a level that is higher relative to the level of protein expressed from a nucleotide sequence encoding the protein that is not codon optimised.

The promoter may be any promoter from which the nucleotide sequences can be transcribed. The coding sequence may be under the control of a constitutive promoter or of a regulatable promoter that initiates transcription only in a particular tissue or cell type, or when the host cell is exposed to some particular stimulus. Typically, the coding sequence is operably linked to a promoter which is not native to the coding sequence. In some embodiments, the promoter expresses the coding sequence in, or is inducible in, tissue in which it is desirable to attract or promote neuronal growth. Examples of promoters which may be used to express nucleic acid sequence in mammalian cells include, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter, a rous sarcoma virus (RSV) promoter, synthetic promoters, hybrid promoters such as cytomegalovirus early enhancer—chicken β-actin 5′ elements—rabbit β-globin splice acceptor element (CAG) and cytomegalovirus early enhancer—chicken β-actin 5′ elements (CBA) and the like. Examples of promoters which express in neurons include synapsin (SYN), calcium/calmodulin-dependent protein kinase (CaMKII), tubulin alpha I (Ta1), neuron-specific enolase (NSE), platelet derived growth factor beta chain (PDGF), MfP, dox, GFAP, Preproenkephalin, dopamine □-hydroxylase (d□H), prolactin, prion protein, murine Thy1.2, myelin basic promoter, or any of the above combined with an enhancer, such as a partial cytomegalovirus promoter. Promoters relevant to muscle selective expression of the neurotropin-encoding DNA include: desmin (Des) promoter, human skeletal muscle α-actin gene (HSA) promoter, muscle creatine kinase (MCK) promoter, and synthetic promoters such as the SPc5-12 promoter. Inducible or controllable promoters include, for example, promoters whose transcriptional activity is modified in the presence or absence of inducers such as mifepristone, doxycycline, tetracycline or tamoxifen.

In one embodiment, the promoter is a constitutive promoter. Examples of constitutive promoters include CMV, SV40, UBC, EF1A, PGK and CAGG promoters.

In one embodiment, the promoter is a CMV promoter.

In one embodiment, the promoter comprises the following nucleotide sequence:

(SEQ ID NO: 7)
aattcgagcttgcatgcctgcaggtcgttacataacttacggtaaatggc
ccgcctggctgaccgcccaacgacccccgcccattgacgtcaataatgac
gtatgttcccatagtaacgccaatagggactttccattgacgtcaatggg
tggagtatttacggtaaactgcccacttggcagtacatcaagtgtatcat
atgccaagtacgccccctattgacgtcaatgacggtaaatggcccgcctg
gcattatgcccagtacatgaccttatgggactttcctacttggcagtaca
tctacgtattagtcatcgctattaccatggtgatgcggttttggcagtac
atcaatgggcgtggatagcggtttgactcacggggatttccaagtctcca
ccccattgacgtcaatgggagtttgttttggcaccaaaatcaacgggact
ttccaaaatgtcgtaacaactccgccccattgacgcaaatgggcggtagg
cgtgtacggtgggaggtctatataagcagagctcgtttagtgaaccgtca
gatcgcctggagacgccatccacgctgttttgacctccatagaagacacc
gggaccgatccagcctccggactctagaggatc

In one embodiment, the nucleotide sequence encoding human BDNF protein, and the nucleotide sequence encoding human NT-3 protein, are operably linked to the same promoter. In an alternative embodiment, the nucleotide sequence encoding human BDNF protein, and the nucleotide sequence encoding human NT-3 protein, are operably linked to independent or separate promoters.

In one embodiment, the recombinant nucleic acid comprises nucleotide sequence encoding BDNF and NT-3 operably linked to a single promoter, wherein:
(a) the promoter is located 5′ to the nucleotide sequence encoding BDNF protein, and an internal ribosome entry site (IRES) is located 3′ to nucleotide sequence encoding the BDNF protein and 5′ to the nucleotide sequence encoding the NT-3 protein; or
(b) the promoter is 5′ to the nucleotide sequence encoding NT-3 protein, and an IRES is located 3′ to nucleotide sequence encoding the NT-3 protein and 5′ to the nucleotide sequence encoding the BDNF protein.

In one embodiment in which the nucleic acid is RNA, the recombinant nucleic acid comprises nucleotide sequence encoding BDNF and NT-3, and:
(a) an internal ribosome entry site (IRES) is located 3′ to nucleotide sequence encoding the BDNF protein and 5′ to the nucleotide sequence encoding the NT-3 protein; or
(b) an IRES is located 3′ to nucleotide sequence encoding the NT-3 protein and 5′ to the nucleotide sequence encoding the BDNF protein.

An internal ribosome entry site (IRES) is a sequence in mRNA which allows for recruitment of eukaryotic ribosomes, and translation initiation of the mRNA that is cap-independent.

In one embodiment, the IRES comprises the following nucleotide sequence:

(SEQ ID NO: 8)
cgcccctctccctcccccccccctaacgttactggccgaagccgcttgga
ataaggccggtgtgcgtttgtctatatgttattttccaccatattgccgt
cttttggcaatgtgagggcccggaaacctggccctgtcttcttgacgagc
attcctaggggtctttcccctctcgccaaaggaatgcaaggtctgttgaa
tgtcgtgaaggaagcagttcctctggaagcttcttgaagacaaacaacgt
ctgtagcgaccctttgcaggcagcggaaccccccacctggcgacaggtgc
ctctgcggccaaaagccacgtgtataagatacacctgcaaaggcggcaca
accccagtgccacgttgtgagttggatagttgtggaaagagtcaaatggc
tctcctcaagcgtattcaacaaggggctgaaggatgcccagaaggtaccc
cattgtatgggatctgatctggggcctcggtgcacatgctttacatgtgt
ttagtcgaggttaaaaaaacgtctaggccccccgaaccacggggacgtgg
ttttcctttgaaaaacacgatgataa.

In one embodiment in which the nucleic acid is DNA, the nucleic acid comprises nucleotide sequence encoding BDNF and NT-3 operably linked to a single promoter, wherein:
(c) the promoter is located 5′ to the nucleotide sequence encoding BDNF protein, and a T2A sequence is located 3′ to nucleotide sequence encoding the BDNF protein and 5′ to the nucleotide sequence encoding the NT-3 protein; or
(d) the promoter is 5′ to the nucleotide sequence encoding NT-3 protein, and a T2A sequence is located 3′ to nucleotide sequence encoding the NT-3 protein and 5′ to the nucleotide sequence encoding the BDNF protein.

T2A is a sequence in mRNA which allows for auto-cleaving at the ribosome to enable production of independent proteins.

In one embodiment in which the nucleic acid is RNA, the recombinant nucleic acid comprises nucleotide sequence encoding BDNF and NT-3, and:
(a) a T2A sequence is located 3′ to nucleotide sequence encoding the BDNF protein and 5′ to the nucleotide sequence encoding the NT-3 protein; or
(b) a T2A sequence is located 3′ to nucleotide sequence encoding the NT-3 protein and 5′ to the nucleotide sequence encoding the BDNF protein.

The nucleic acid molecule described herein typically comprises a polyadenylation signal. In one embodiment, the polyadenylation signal is a bovine growth hormone polyadenylation signal. In embodiments in which the nucleic acid is DNA, the bovine growth hormone polyadenylation signal comprises the following sequence:

(SEQ ID NO: 9)
cgctgatcagcctcgactgtgccttctagttgccagccatctgttgtttg
cccctcccccgtgccttccttgaccctggaaggtgccactcccactgtcc
tttcctaataaaatgaggaaattgcatcgcattgtctgagtaggtgtcat
tctattctggggggtggggggggcaggacagcaagggggaggattgggaa
gacaatagcaggcatgctggggatgcggtgggctctatggcttctgaggc
ggaaagaaccagctgggg.

In one embodiment, the recombinant nucleic acid is DNA and comprises in order from 5′ to 3′:
(a) a promoter;
(b) nucleotide sequence encoding BDNF protein;
(c) an internal ribosome entry site (IRES);
(d) nucleotide sequence encoding NT-3 protein; and
(e) a polyadenylation signal.

In one embodiment, the recombinant nucleic acid is DNA and comprises the following nucleotide sequences (a) to (f):

(a) CMV promoter:
aattcgagcttgcatgcctgcaggtcgttacataacttacggtaaatggc
ccgcctggctgaccgcccaacgacccccgcccattgacgtcaataatgac
gtatgttcccatagtaacgccaatagggactttccattgacgtcaatggg
tggagtatttacggtaaactgcccacttggcagtacatcaagtgtatcat
atgccaagtacgccccctattgacgtcaatgacggtaaatggcccgcctg
gcattatgcccagtacatgaccttatgggactttcctacttggcagtaca
tctacgtattagtcatcgctattaccatggtgatgcggttttggcagtac
atcaatgggcgtggatagcggtttgactcacggggatttccaagtctcca
ccccattgacgtcaatgggagtttgttttggcaccaaaatcaacgggact
ttccaaaatgtcgtaacaactccgccccattgacgcaaatgggcggtagg
cgtgtacggtgggaggtctatataagcagagctcgtttagtgaaccgtca
gatcgcctggagacgccatccacgctgttttgacctccatagaagacacc
gggaccgatccagcctccggactctagaggatc
(b) NotI restriction site (‘gcggccgc’)
followed by a Kozak sequence:
gcggccgcgccacc
(c) BDNF sequence:
atgactatcctcttcctcacaatggtcatcagctacttcggatgtatgaa
agctgcacctatgaaggaagccaacattagaggacaaggaggactcgcct
atcctggagtcagaactcacggtactctggagagcgtcaatggtccaaag
gctggaagcagaggtctgaccagcctggctgataccttcgagcacgttat
cgaggaactcctggatgaggaccagaaggttaggccaaatgaagagaaca
acaaagatgctgacctgtacacttctcgcgtgatgctcagctctcaagtt
ccactggagcctccactcctgttcctcctggaggaatacaagaactacct
ggatgctgccaacatgtccatgagagttagaaggcactccgatccagctc
gcagaggtgaactctctgtctgcgatagcatctctgagtgggtcaccgct
gctgacaagaagacagctgtggacatgagcggtggaacagtgaccgtgct
ggagaaggtcccagtctctaagggtcaactgaagcagtacttctacgaaa
caaagtgcaacccaatgggctatactaaggagggttgtagaggtattgat
aagagacattggaacagccagtgcagaacaactcaatcttatgttagggc
tctgactatggactccaagaagaggattggttggaggttcatcaggatcg
acacttcctgcgtgtgtacactgacaatcaagagaggacgctag
(d) IRES sequence:
cgcccctctccctcccccccccctaacgttactggccgaagccgcttgga
ataaggccggtgtgcgtttgtctatatgttattttccaccatattgccgt
cttttggcaatgtgagggcccggaaacctggccctgtcttcttgacgagc
attcctaggggtctttcccctctcgccaaaggaatgcaaggtctgttgaa
tgtcgtgaaggaagcagttcctctggaagcttcttgaagacaaacaacgt
ctgtagcgaccctttgcaggcagcggaaccccccacctggcgacaggtgc
ctctgcggccaaaagccacgtgtataagatacacctgcaaaggcggcaca
accccagtgccacgttgtgagttggatagttgtggaaagagtcaaatggc
tctcctcaagcgtattcaacaaggggctgaaggatgcccagaaggtaccc
cattgtatgggatctgatctggggcctcggtgcacatgctttacatgtgt
ttagtcgaggttaaaaaaacgtctaggccccccgaaccacggggacgtgg
ttttcctttgaaaaacacgatgataa
(e) Swal restriction site (‘atttaaat’)
followed by a Kozak element:
agatttaaatcacc
(f) NT-3 sequence:
atgtctatcctgttctatgtgatcttcctcgcatacctgagaggaatcca
aggcaacaacatggatcaacgcagcctgccagaagacagcctgaactctc
tgatcatcaagctcatccaagcagacattctgaagaacaagctgtccaaa
cagatggtggatgtcaaagagaactatcagtccactctgcccaaagcaga
ggctcccagggagccagaaagaggtggacctgccaagtctgctttccaac
cagtgattgcaatggacactgagctgctcaggcagcagaggaggtacaat
tctcccagggttctgctctctgattccactcctctggaaccaccacctct
ctacctgatggaggattacgtgggttctccagtggtggcaaacagaacaa
gcagacgcaagaggtatgcagaacacaaatcccacagaggtgagtatagc
gtgtgcgactctgaatctctgtgggtgactgacaaatcctctgctattga
cattagaggacatcaggtgacagtgctcggagagatcaagactggcaata
gcccagtcaaacaatacttctacgaaactcgctgtaaagaggcaaggcca
gttaagaacggttgtagaggtatcgacgacaaacactggaatagccagtg
taagacttctcaaacctacgttagagccctcacatctgagaacaacaagc
tcgtcggatggaggtggatcagaattgatacctcctgcgtctgtgcactg
agcaggaagattggaagaacctaa
(g) Bovine growth hormone poly(A) signal:
(SEQ ID NO: 10)
cgctgatcagcctcgactgtgccttctagttgccagccatctgttgtttg
cccctcccccgtgccttccttgaccctggaaggtgccactcccactgtcc
tttcctaataaaatgaggaaattgcatcgcattgtctgagtaggtgtcat
tctattctggggggtggggggggcaggacagcaagggggaggattgggaa
gacaatagcaggcatgctggggatgcggtgggctctatggcttctgaggc
ggaaagaaccagctgggg

The DNA sequences described herein may be stand alone or may be part of a vector.

In one aspect, there is provided a vector comprising the recombinant nucleic acid described herein. The term “vector” refers to a nucleic acid sequence suitable for transferring nucleic acid into a host cell, such as a mammalian cell. The term “vector” includes, for example, plasmids, cosmids, etc., which may be used to produce mRNA. In one embodiment, the vector is a plasmid vector. A plasmid vector is a double stranded circular DNA molecule into which additional sequence may be inserted. Plasmids are known in the art and described in, for example, Sambrook et al. Molecular Cloning: A Laboratory Manual, 4^{th} Ed. Vol. 1-3, Cold Spring Harbor, N.Y. (2012).

In one embodiment, the nucleic acid is RNA and comprises the following nucleotide sequence:

(SEQ ID NO: 15)
augacuauccucuuccucacaauggucaucagcuacuucggauguaugaa
agcugcaccuaugaaggaagccaacauuagaggacaaggaggacucgccu
auccuggagucagaacucacgguacucuggagagcgucaaugguccaaag
gcuggaagcagaggucugaccagccuggcugauaccuucgagcacguuau
cgaggaacuccuggaugaggaccagaagguuaggccaaaugaagagaaca
acaaagaugcugaccuguacacuucucgcgugaugcucagcucucaaguu
ccacuggagccuccacuccuguuccuccuggaggaauacaagaacuaccu
ggaugcugccaacauguccaugagaguuagaaggcacuccgauccagcuc
gcagaggugaacucucugucugcgauagcaucucugagugggucaccgcu
gcugacaagaagacagcuguggacaugagcgguggaacagugaccgugcu
ggagaaggucccagucucuaagggucaacugaagcaguacuucuacgaaa
caaagugcaacccaaugggcuauacuaaggaggguuguagagguauugau
aagagacauuggaacagccagugcagaacaacucaaucuuauguuagggc
ucugacuauggacuccaagaagaggauugguuggagguucaucaggaucg
acacuuccugcguguguacacugacaaucaagagaggacgcgagggcaga
ggaagucugcuaacaugcggtgacgucgaggagaauccuggcccaauguc
uauccuguucuaugugaucuuccucgcauaccugagaggaauccaaggca
acaacauggaucaacgcagccugccagaagacagccugaacucucugauc
aucaagcucauccaagcagacauucugaagaacaagcuguccaaacagau
gguggaugucaaagagaacuaucaguccacucugcccaaagcagaggcuc
ccagggagccagaaagagguggaccugccaagucugcuuuccaaccagug
auugcaauggacacugagcugcucaggcagcagaggagguacaauucucc
caggguucugcucucugauuccacuccucuggaaccaccaccucucuacc
ugauggaggauuacguggguucuccagugguggcaaacagaacaagcaga
cgcaagagguaugcagaacacaaaucccacagaggugaguauagcgugug
cgacucugaaucucugugggugacugacaaauccucugcuauugacauua
gaggacaucaggugacagugcucggagagaucaagacuggcaauagccca
gucaaacaauacuucuacgaaacucgcuguaaagaggcaaggccaguuaa
gaacgguuguagagguaucgacgacaaacacuggaauagccaguguaaga
cuucucaaaccuacguuagagcccucacaucugagaacaacaagcucguc
ggauggagguggaucagaauugauaccuccugcgucugugcacugagcag
gaagauuggaagaaccuaa

One aspect provides an isolated nucleic acid comprising the nucleotide sequence of SEQ ID NO: 15.

In another aspect, there is provided a pharmaceutical composition comprising the nucleic acid described herein. The composition comprises the nucleic acid in a pharmaceutically acceptable carrier. Methods for the formulation of agents with pharmaceutical carriers are known in the art and are described in, for example, Remington's Pharmaceutical Science, (17th ed. Mack Publishing Company, Easton, Pa. 1985); Goodman & Gillman's: The Pharmacological Basis of Therapeutics (11th Edition, McGraw-Hill Professional, 2005).

Acceptable carriers, diluents and adjuvants are nontoxic to recipients, are preferably inert at the dosages and concentrations employed, and are typically amendable to electroporation.

The pharmaceutically acceptable carrier can be a solvent or dispersion medium containing, for example, water, ethanol, polyol (for example, glycerol, propylene glycol, liquid polyethylene glycol and the like), sucrose and other polysaccharoses or saccharides, suitable mixtures thereof, and vegetable oils.

In one embodiment, the pharmaceutical composition comprises the nucleic acids or vectors described herein, and a pharmaceutically acceptable carrier that has low electrical conductivity. This low conductivity supports ‘conductivity-clamping’ in the vicinity of the gene electrotransfer probe, enhancing the electric field strength and biasing the current to the polyanionic nucleic acid molecules, thereby achieving enhanced translocation of the therapeutic molecules to the targeted cell membranes. Typically, the pharmaceutically acceptable carrier that has low electrical conductivity is iso-osmotic with respect to extracellular fluid. An example of a pharmaceutically acceptable carrier that has low electrical conductivity is a polysaccharose or a saccharide. As used herein, a “polysaccharose” is any of a class of carbohydrates, such as sucrose, starch or cellulose, consisting of a number of monosaccharides joined by glycosidic bonds. In one embodiment, the polysaccharose is sucrose. In one embodiment, the composition comprises sucrose in solution at a concentration in the range of from about 5-15% w/v, typically 6-14% w/v, 7-13% w/v, 8-12% w/v, 9-12% w/v, or 9-11% w/v. In one embodiment, the concentration of sucrose is about 10% w/v. In another embodiment, the pharmaceutically acceptable carrier that has low conductivity comprises a radio-opaque contrast agent. Use of a radio-opaque contrast agent enables visualization of the delivery of the DNA/RNA within the target tissue while achieving optimum electrotransfer efficiency by supporting local conductivity clamping. Use of the radio-opaque contrast agent provides local control of the conductivity to maximize field strength during electrotransfer and further promotes translocation of the polyanionic nucleic acid molecules.

The nucleic acids or vectors described herein may be formulated for introduction into cells by electroporation. Although any electroporation or electrotransfer method and system may be used. Advantageous electrotransfer systems and methods are described in WO2016/205895, WO2014/201511, WO2011/006204, WO2020/118383 and WO2021/072507.

A further aspect provides methods of increasing BDNF and/or NT-3 protein expression in a mammalian cell, comprising introducing into the cell the recombinant nucleic acid molecule described herein, or the vector described herein.

A further aspect concerns the incorporation of synthetic elements to ribonucleic (RNA) nucleotides to enhance stability of the molecules. Examples include the incorporation of 5-methoxyuradine-5′-triphosphate to reduce immunogenicity and other nucleoside-modified messenger RNA (modRNA) where naturally occurring nucleosides are substituted for naturally modified nucleosides or synthetic nucleoside analogues.

Another aspect provides a method of promoting neuron growth in mammalian tissue, comprising introducing into cells of the tissue the recombinant nucleic acid molecule described herein, the vector described hereinor the composition described herein.

In one embodiment, recombinant nucleic acid described herein may be introduced into the mesenchymal cells lining the scala tympani perilymphatic space of the cochlea to promote growth of auditory nerve fibres. Such an approach may regrow auditory nerve fibres and direct that regrowth to the vicinity of a cochlear implant electrode array, which improves the neural interface with a cochlear hearing prosthesis. In this embodiment, the recombinant nucleic acid is delivered to the mesenchymal cells lining the scala tympani perilymphatic space of the cochlea. This transforms these cells into neurotrophin secreting cells.

Accordingly, one embodiment provides a method of promoting auditory nerve fibre growth in cochlear tissue, comprising introducing into cells of the cochlear tissue the recombinant nucleic acid molecule described herein, the vector described herein, or the composition described herein.

One embodiment provides a method of promoting auditory nerve fibre growth in cochlear tissue, comprising introducing into mesenchymal cells lining the scala tympani perilymphatic space of the cochlear tissue the recombinant nucleic acid molecule described herein, the vector described herein, or the composition described herein.

The inventors further envisage that the nucleic acid molecules described herein may be used to promote nerve repair following trauma, or to promote neuronal growth and/or survival in nerve transplantation. In such applications, the nucleic acid may be delivered to the target muscle tissue to promote the innervation of the target tissue with transplanted nerve fibres.

Another embodiment provides a method of promoting growth and/or survival of transplanted neurons in tissue, comprising introducing into cells of the tissue that has been, or is to be, transplanted with nerves, the recombinant nucleic acid molecule described herein, the vector described herein, or the composition described herein.

The inventors envisage that the recombinant nucleic acid molecule described herein may be used in the treatment of spinal cord injury, where gene augmentation therapy using this gene construct may promote repair of the spinal cord.

Another embodiment therefore provides a method of promoting growth and/or survival of neurons in spinal cord, comprising introducing into cells of spinal cord tissue the recombinant nucleic acid molecule described herein, the vector described herein, or the composition described herein.

The inventors envisage that the recombinant nucleic acid molecule described herein may be used to promote outgrowth of retinal ganglion neurons, for example, towards an implanted electrode array.

Accordingly, a further embodiment provides a method of promoting growth of retinal ganglion neurons, comprising introducing into the retinal ganglion neurons, or associated glia or connective tissue, the nucleic acid molecule described herein, the vector described herein, or the composition described herein. In one embodiment, the growth is directed towards an implanted electrode array.

The inventors further envisage that the nucleic acid molecule described herein may be used in deep brain stimulation, where use of the recombinant nucleic acid molecule described herein in conjunction with the deep brain stimulator electrode array may improve the local neural interface, lowering the current stimulus levels required to sustain treatment of Parkinson's Disease or other neurological and psychological disorders with a focal neural etiology.

The term “administering” should be understood to mean providing a compound or agent to a subject in need of treatment.

It will be understood that the specific dose level and frequency of dosage for any particular subject may be varied and will depend upon a variety of factors including, for example, the activity of the specific compound or agent employed, the metabolic stability and length of action of that compound or agent, the age, body weight, general health, sex, diet, mode and time of administration, drug combination, the severity of the particular condition, and the host undergoing therapy.

Also provided is a kit, comprising a container comprising the agent. The container may be simply a bottle comprising the agent in parenteral dosage form, each dosage form comprising a unit dose of the agent. The kit will further comprise printed instructions. The article of manufacture will comprise a label or the like, indicating treatment of a subject according to the present method. In one form, the article of manufacture may be a container comprising the agent in a form for parenteral dosage. For example, the agent may be in the form of an injectable solution in a disposable container.

As used herein, “treating” means affecting a subject, tissue or cell to obtain a desired pharmacological and/or physiological effect and includes inhibiting the condition, i.e. arresting its development; or relieving or ameliorating the effects of the condition i.e. cause reversal or regression of the effects of the condition.

As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In order to exemplify the nature of the present invention such that it may be more clearly understood, the following non-limiting examples are provided.

### EXAMPLES

### Intramuscular BaDGE® DNA/mRNA Electrotransfer

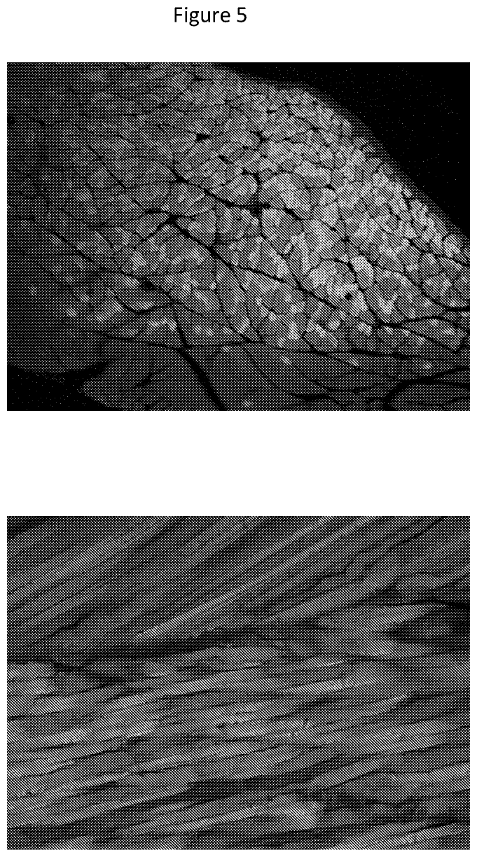

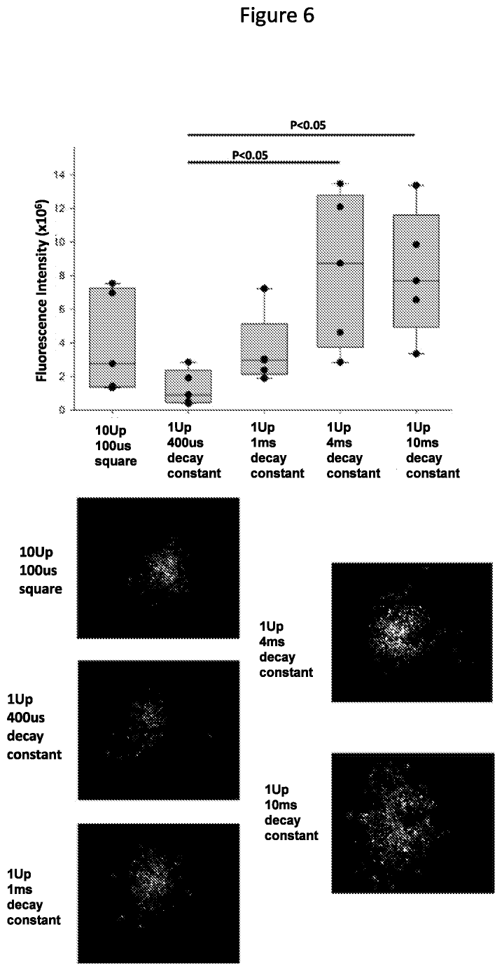

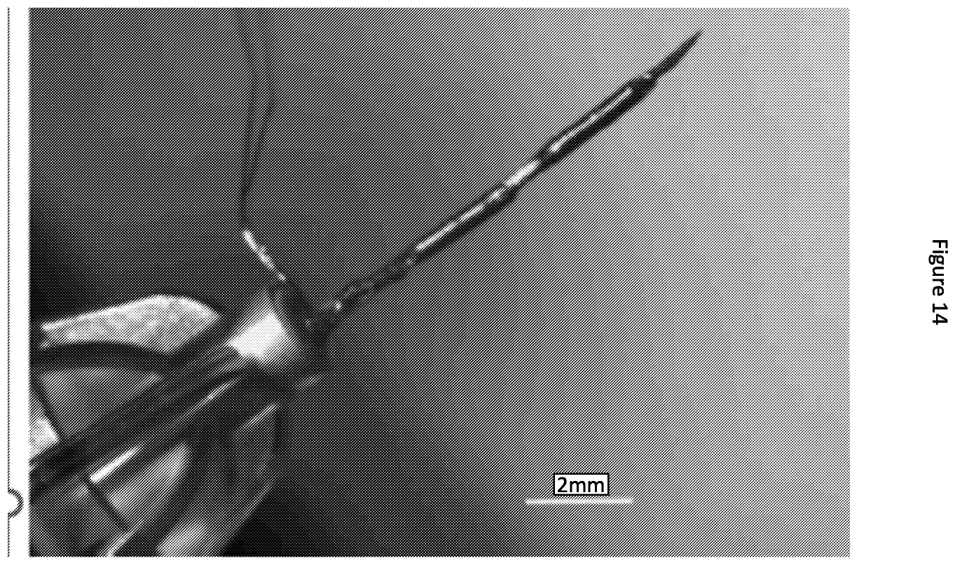

Method: Under an approved animal ethics protocol (UNSW ACEC), isoflurane anaesthesia (4% induction, 2% maintenance), hindlimbs of adult female BALB/cJAus mice were shaved. Firefly luciferase encoding DNA or mRNA were delivered into mouse hindlimb tissue by single capacitive discharge gene electrotransfer as shown in FIG. 5, using a probe as shown in FIG. 14, and as described in WO2021/072507. Firefly luciferase encoding DNA (0.5 or 2 μg/μl) or mRNA (0.5 μg/μl) in BaDGE®-carrier® solution (iso-osmotic (10%) sucrose) were loaded into the BaDGE®-delivery device (BaDGE® BEPv1 electrotransfer probe; BaDGE® Single Capacitive Discharge control unit 2 (BSCDcu2) or delivery probe (part ref. BSCDp1) and docking station (part ref. BSCDds1)) with a low deadspace syringe until a satellite drop appeared at the tip of the BaDGE®-probe. The BaDGE®-probe was then inserted into the BaDGE®-docking station to charge the capacitor in the BaDGE®-probe hub (2.2 μF, 120 V or 200 V). The charged BaDGE® needle was then inserted into the mouse hindlimb muscle (incl. gastrocnemius muscle) to the point of transition between the first and second electrode and 30 μl of the nucleic acids in BaDGE®-carrier® solution injected before the needle further inserted to the point where the second electrode was completely inserted into the muscle, leaving the injected nucleic acid BaDGE®-carrier® solution in the centre of the electric field lens (between the two concentric electrodes). A single capacitive discharge was then delivered by pressing the discharge button on the hub of the BaDGE®-probe®. The needle was then removed, and the mouse returned to the home cage after recovery. For bioluminescence imaging mice underwent repeated isoflurane anaesthesia (4% induction, 2% maintenance), hindlimbs shaved if required and luciferin (150 mg/kg in normal saline) intraperitoneally injected. The mouse was then placed into the IVIS Spectrum CT and total photon flux within a set region of interest over each hindlimb measured every minute until peak bioluminescence passed with signal dropping for at least 5 consecutive measurements.

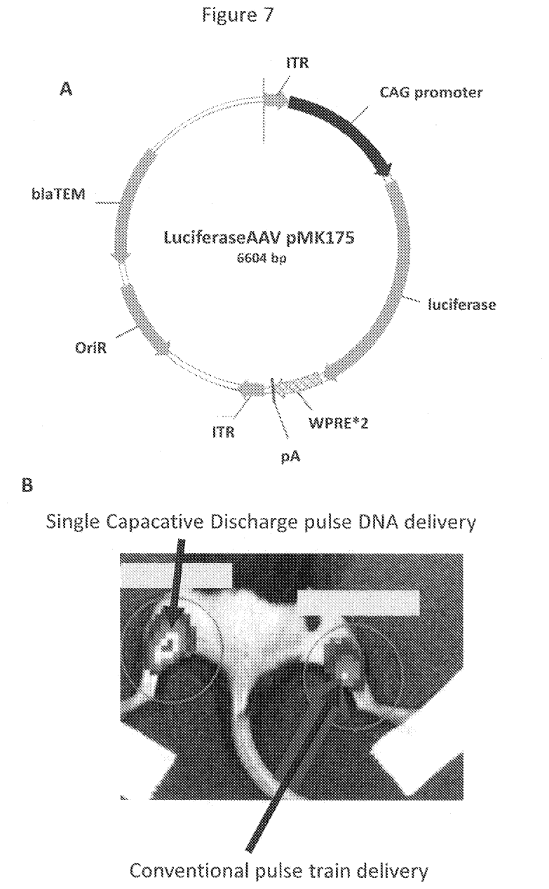

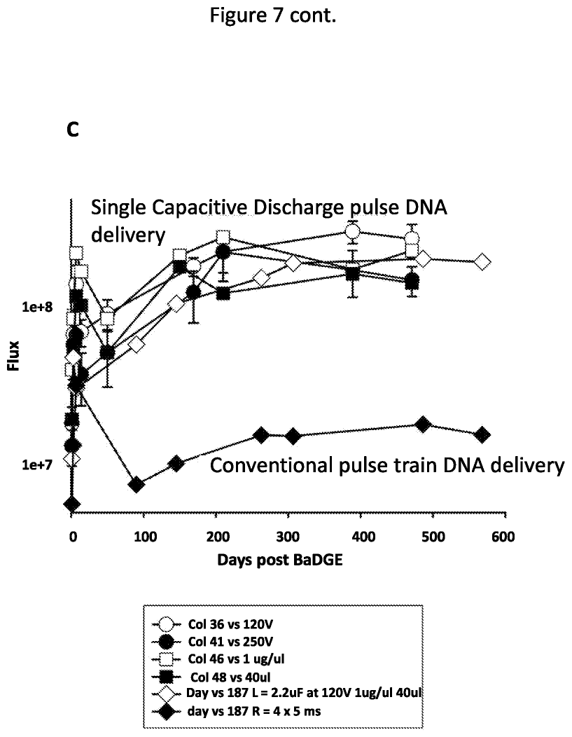

A first set of experimental data comparing in vivo gene expression in mouse hind-limb muscle where luciferase reporter plasmid DNA was delivered via BaDGE is shown in FIG. 7 and as described in WO2021/072507, comparing conventional square wave pulse train vs. single capacitive discharge (SCD); plus circles open and closed are 2 voltages SCD from 3 mice—open square—vs closed square (40 μl)—3 mice/legs, with plasmid DNA delivered in 10% sucrose solution; Utilizes BEPv1 electrotransfer probe and Single Capacitive Discharge control unit 2 (BSCDcu2/UNSWref1032) with discrete capacitor charging. —4 mice in total. Repeated bioluminescence measurements in vivo out to 471 days for most of the mice and 568 days for one of the mice.

These data show that SCD works very efficiently, expression is indefinite (study terminated at 568 days post BaDGE with sustained expression)—for BaDGE irrespective of pulse train or SCD, and volume and voltage for SCD are broadly tolerant.

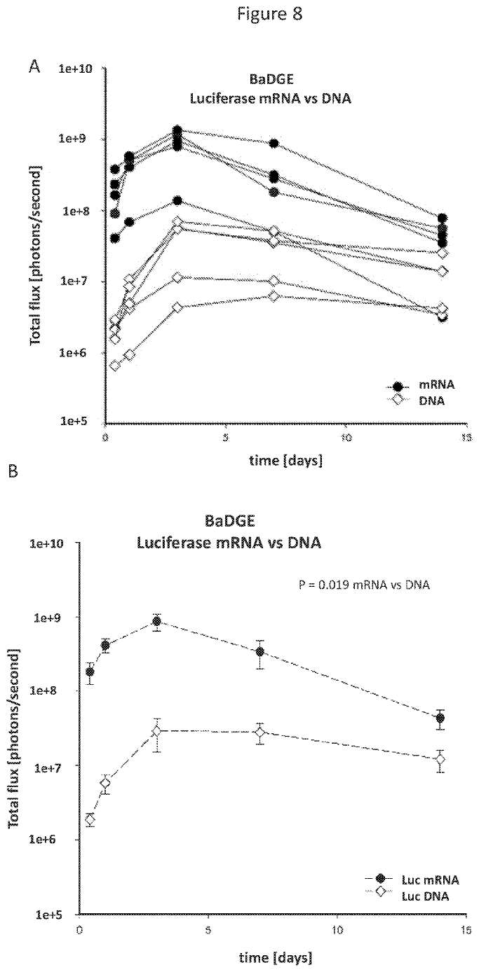

In vivo expression levels of luciferase protein was then tested following delivery of DNA or mRNA encoding luciferase using BEPv1 electrotransfer probe and Single Capacitive Discharge control unit, and the results compared. Results are show in FIG. 8.

The results show higher luciferase protein-mediated bioluminescence following mRNA delivery over pDNA from the first measurement at 10 hours, out to 14 days (data shown for 10 hours, 1, 3, 7 & 14 days). FIG. 8A. is Longitudinal data across individual legs. FIG. 8B. is Average data (mean±s.e.m.); repeated measures two way ANOVA comparing mRNA vs DNA P=0.019; n=5 per group.

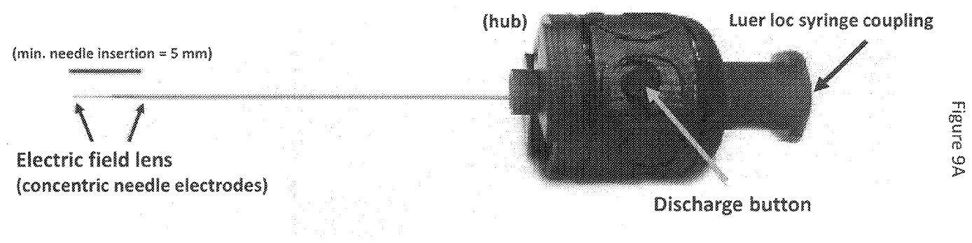

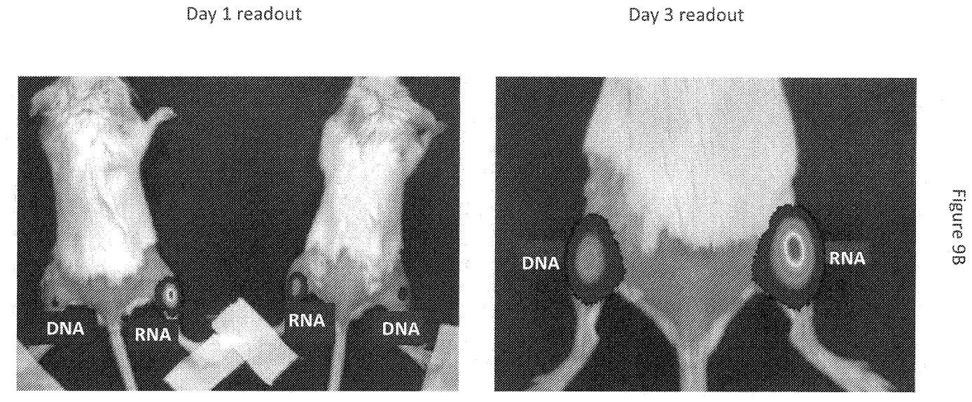

A further study evaluated the onset kinetics and longevity of mRNA vs plasmid DNA encoding the luciferase reporter gene delivered by single capacitive discharge to the mouse hindlimb muscle, with an earlier initial census point (3 hours), utilising a refined single capacitive discharge (SCD) BaDGE® muscle delivery system (BaDGE® BSCDp1 delivery probe and docking station (part ref. BSCDds1). The probe and charging station used was as disclosed in WO 2021/72507. The probe is shown in FIG. 9A.

FIG. 9B shows enhanced expression from mRNA over DNA from 1 (ref. ms69 & ms70) and 3 days (ref. ms66) (BALB/c mouse strain). Signal is readout of bioluminescence intensity following intraperitoneal injection of the luciferin substrate, measured using a IVIS Spectrum CT imaging platform.

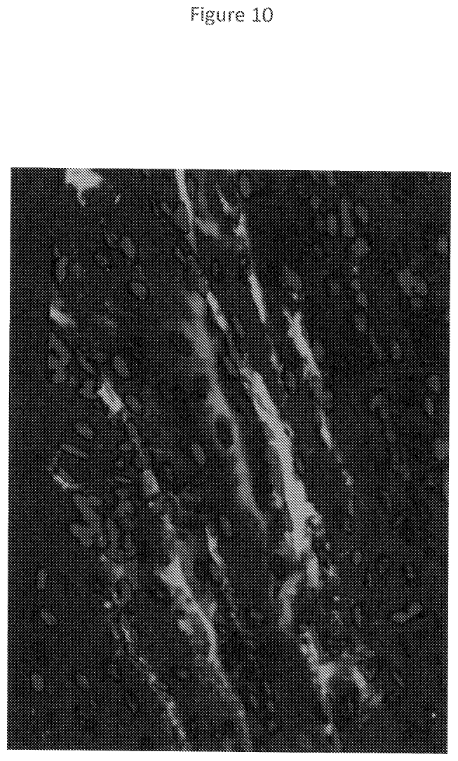

The expression of luciferase protein in the hindlimb muscle was confirmed using immunohistochemistry, which is shown in FIG. 10. Fixed tissue was cryosectioned and immunolabelled using an anti-luciferase antibody.

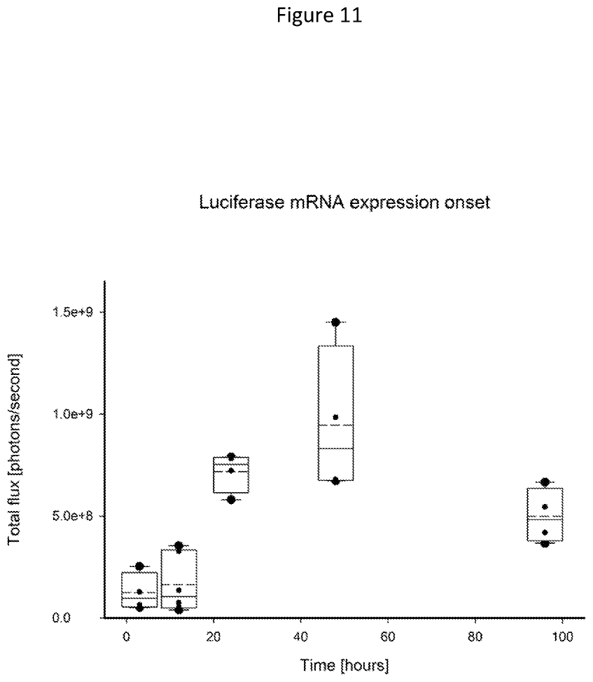

Luc-mRNA expression onset in mouse hindlimb in vivo (luciferase bioluminescence) was undertaken with single measurements for 3 h and 12 h time points; 24 h time point measurements were re-measured at 48 h and 96 h. The results are shown in FIG. 11.

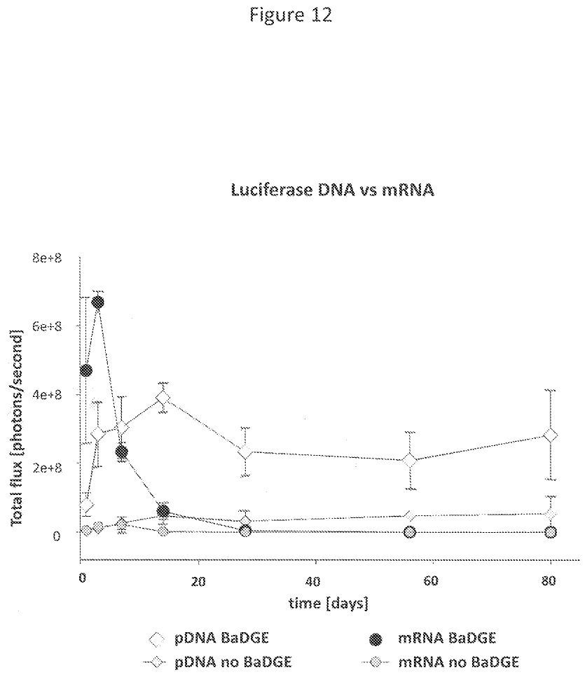

In a further study, recombinant luciferase protein onset and decay was compared after either mRNA or DNA delivery to the adult BALB/cJ mouse hindlimbs in vivo. Luciferase bioluminescence was measured in hindlimbs bilaterally, following randomised delivery of luc-mRNA to one hindlimb of each mouse, and delivery of luc-DNA to the contralateral hindlimb. BaDGE® electrotransfer and control treatment groups (no electrotransfer) were included for mRNA and DNA delivery. The results are shown in FIG. 12.

### Optimized Human Brain-Derived Neurotrophic Factor (BDNF) and Neurotrophin-3 (NT3) Expression Cassette

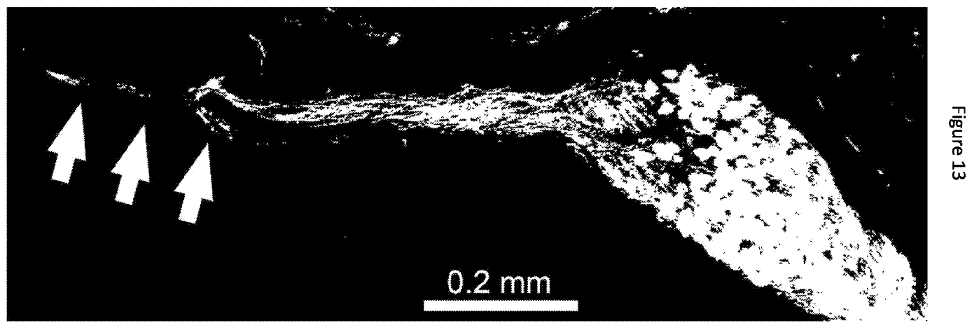

Example of development and validation of an Optimized Human Brain-derived neurotrophic factor (BDNF) and Neurotrophin-3 (NT3) Expression Cassette (OHBNEC-CMVp-BDNF-IRES-NT3) suitable for expression in mammalian tissues or for production of mRNA. Production of a OHBNEC includes:
1. fully synthetic manufacture of OHBNEC-CMVp-BDNF-IRES-NT3 by base-wise nucleoside incorporation of the BDNF-IRES-NT-3 expression cassette, with 5′ CMV promoter sequence and 3′ Bovine poly(A) signal, packaged into a plasmid vector (pClonEZ) using custom DNA production service (Epoch LifeSciences, USA).
2. Heat-shock transformation of E. coli cell line (Top10-Invitrogen) with OHBNEC-CMVp-BDNF-IRES-NT3, with plasmid purification using ion affinity chromatography
3. BaDGE® electrotransfer of the OHBNEC-CMVp-BDNF-IRES-NT3 plasmid into human embryonic kidney cell line 293 (HEK293) cells with sampling of the supernatant for ELISA analysis of BDNF and NT-3 protein production by HEK293 cells (See Table. 1)
4. Proof of concept in vivo efficacy neurotrophin activity using an auditory nerve fibre regeneration model (guinea pig chemically-induced cochlear neuropathy). FIG. 13 shows image of guinea pig cochlear spiral ganglion neuron regeneration following BaDGE® electrotransfer of OHBNEC-CMVp-BDNF-IRES-NT3 plasmid.

TABLE 1

| | | |
|---|---|---|
| | | Increase |
| | | due to |
| | | biphasic |
| Monophasic | Biphasic | condition |
| BaDGE ® | BaDGE ® | (percentage |
| electrotransfer | electrotransfer | change) |

Comparison of HEK293 cell mCherry expression across CMVp-
mCherry(nis) plasmid DNA BaDGE ® electrotransfer conditions
(mean ± s.e.m., n = 6); p = 0.00018 (t-test)

| | | | |
|---|---|---|---|
| HEK293 cell | 123.8 ± 22.8 | 431.3 ± 118.4 | 307.5 |
| mCherry | | | (248%) |
| sum pixel | | | |
| intensity | | | |

Comparison of BDNF & NT3 protein levels across OHBNEC-
CMVp-BDNF-IRES-NT3 plasmid BaDGE ® electrotransfer
conditions
(mean ± s.e.m., n = 6); p = 0.001 (ANOVA)

| | | | |
|---|---|---|---|
| BDNF | 141 ± 11.3 | 218 ± 13.1 | 77.0 |
| Concentration | | | (55%) |
| (pg/ml) | | | |
| NT3 | 6.3 ± 5.3 | 13.5 ± 3.6 | 7.2 |
| Concentration | | | (114%) |
| (pg/ml) | | | |

Table 1. Demonstration of production of recombinant BDNF and NT-3 neurotrophin proteins by HEK293 cells following BaDGE ® electrotransfer of the OHBNEC- CMVp-BDNF-IRES-NT3 plasmid. HEK293T cells (Merck) were plated onto coverslips and maintained in an incubator (5% CO₂ at 37 °C for 1 day in DMEM (Sigma) with 5% foetal calf serum (FCS; Sigma). The coverslips were then removed to an electrotransfer stage and a cocktail of the OHBNEC plasmid and an mCherry reporter plasmid was overlaid on the cells and a close-field pulsed-electric field was used to transfect the cells (BaDGE ®, after Pinyon et al, 2014). Two different electrotransfer conditions were applied (monophasic and biphasic) and the differential expression was analysed. The coverslips were returned to culture and maintained for four days prior to imaging the mCherry fluorescence and sampling of the supernatant for ELISA quantification of BDNF and NT-3 protein levels.

### Expression of BDNF and NT3 in Cochlear of Guinea Pigs

An mRNA molecule encoding BDNF and NT3 with the T2A bicistronic element for dual expression was delivered in vivo to the cochlea of guinea pigs via the BaDGE gene delivery array, using conductivity-clamped gene electrotransfer using an alternating capacitive discharge—driven focused electric field. The cochlear perilymph was subsequently sampled and the concentrations of the neurotrophins compared between treated and untreated cochleae. The Data shows significant production of the (recombinant) neurotrophins over the baseline levels (untreated cochleae).

### Methods

Coloured male guinea pigs were anaesthetized with isoflurane and analgesia provided (buprenorphine 0.025 mg/kg, medetomidine 0.15 mg/kg, and lignocaine 0.2 ml of 2% solution at the surgical site). Under sterile conditions, a unilateral postauricular skin incision was made and a small hole drilled through the bulla exposing the round window. The round window membrane was perforated and the clinical BaDGE® gene delivery array inserted into scala tympani. The scala was then perfused with 30 μl BDNF-T2A-NT3 mRNA (0.5 μg/μl in iso-osmotic 10% sucrose solution) at a flow rate of 10 μl/min controlled via a peristaltic syringe pump and electrotransfer carried out via capacitive discharge (@ 200V) through the clinical BaDGE® gene delivery array as described in WO 2021/72507. The nucleotide sequence of BDNF-T2A-NT3 is as follows:

(SEQ ID NO: 15)
augacuauccucuuccucacaauggucaucagcuacuucggauguaugaa
agcugcaccuaugaaggaagccaacauuagaggacaaggaggacucgccu
auccuggagucagaacucacgguacucuggagagcgucaaugguccaaag
gcuggaagcagaggucugaccagccuggcugauaccuucgagcacguuau
cgaggaacuccuggaugaggaccagaagguuaggccaaaugaagagaaca
acaaagaugcugaccuguacacuucucgcgugaugcucagcucucaaguu
ccacuggagccuccacuccuguuccuccuggaggaauacaagaacuaccu
ggaugcugccaacauguccaugagaguuagaaggcacuccgauccagcuc
gcagaggugaacucucugucugcgauagcaucucugagugggucaccgcu
gcugacaagaagacagcuguggacaugagcgguggaacagugaccgugcu
ggagaaggucccagucucuaagggucaacugaagcaguacuucuacgaaa
caaagugcaacccaaugggcuauacuaaggaggguuguagagguauugau
aagagacauuggaacagccagugcagaacaacucaaucuuauguuagggc
ucugacuauggacuccaagaagaggauugguuggagguucaucaggaucg
acacuuccugcguguguacacugacaaucaagagaggacgcgagggcaga
ggaagucugcuaacaugcggtgacgucgaggagaauccuggcccaauguc
uauccuguucuaugugaucuuccucgcauaccugagaggaauccaaggca
acaacauggaucaacgcagccugccagaagacagccugaacucucugauc
aucaagcucauccaagcagacauucugaagaacaagcuguccaaacagau
gguggaugucaaagagaacuaucaguccacucugcccaaagcagaggcuc
ccagggagccagaaagagguggaccugccaagucugcuuuccaaccagug
auugcaauggacacugagcugcucaggcagcagaggagguacaauucucc
caggguucugcucucugauuccacuccucuggaaccaccaccucucuacc
ugauggaggauuacguggguucuccagugguggcaaacagaacaagcaga
cgcaagagguaugcagaacacaaaucccacagaggugaguauagcgugug
cgacucugaaucucugugggugacugacaaauccucugcuauugacauua
gaggacaucaggugacagugcucggagagaucaagacuggcaauagccca
gucaaacaauacuucuacgaaacucgcuguaaagaggcaaggccaguuaa
gaacgguuguagagguaucgacgacaaacacuggaauagccaguguaaga
cuucucaaaccuacguuagagcccucacaucugagaacaacaagcucguc
ggauggagguggaucagaauugauaccuccugcgucugugcacugagcag
gaagauuggaagaaccuaa

24 hours (n=1) or 1 week (n=3) guinea pigs were euthanised and the perilymph collected from the treated and the untreated ear. Neurotrophin protein levels were measured using a BDNF or NT3 DuoSet™ ELISA (R&D Systems) according to the manufacturer's instructions. Briefly, standard polystyrene 96 well plates were coated with capture antibody overnight, blocked with reagent dilutant and BDNF standard (1500-23.4 μg/ml for BDNF and 2000-31.3 μg/ml for NT3), or guinea pig perilymph sample (1:50 in reagent dilutant) applied. Following 3 washes, the biotinylated detection antibody was added followed by Streptavidin-HRP. A colorimetric reaction was initiated, stopped and absorbance measured at 405 nm and 530 nm using a FlexStation 3 Microplate Reader and the BDNF and NT3 concentrations calculated against the relevant standard curve. Undertaken with UNSW Animal Care and Ethics Committee approval.

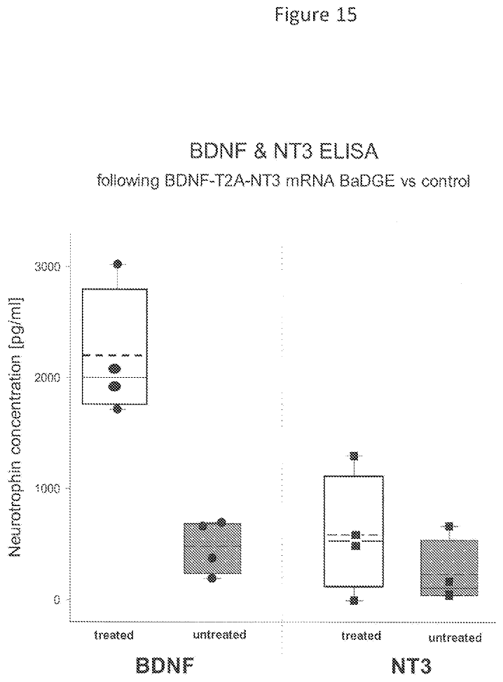

The results are shown in FIG. 15. The data shows a significant effect of the treatment (P<0.001). The data shows a greater than 4-fold increase of BDNF level in the treated (2173.9±290.1 pg/ml) over the untreated (479.4±120.2 pg/ml) control perilymph (P<0.01). The NT3 mean level in the treated (590.3±268.6 pg/ml) was doubled over the untreated (231.2±147.2 pg/ml) control perilymph.

The results show that following electrotransfer of mRNA encoding BDNF and NT3 into the cochlear, there is significant production of BDNF and NT3 over the baseline levels of untreated cochleae.

1-57. (canceled)

58. A method of expressing and/or increasing expression of a protein of interest in one or more cells of tissue in a target region, the method comprising:
(a) introducing to the tissue in the target region RNA comprising nucleotide sequence capable of expressing the protein of interest in the one of more cells of the tissue; and
(b) inducing via two or more electrodes proximate the target region an electric potential difference in the tissue of the target region, the electric potential difference causing an electric field through the target region, to thereby promote entry of the RNA into the one or more cells of the tissue.

59. The method of claim 58, wherein the RNA is naked mRNA.

60. The method of claim 58, wherein the RNA comprises one or more modified nucleotides.

61. The method of claim 58, wherein the RNA encodes more than one protein.

62. The method of claim 58, wherein the protein is expressed within 4 hours of applying the electric field.

63. The method of claim 58, wherein the protein is expressed within 2 hours of applying the electric field.

64. The method of claim 58, wherein the RNA comprises a nucleotide sequence encoding human BDNF protein or comprises nucleotide sequence encoding the human BDNF protein and human NT-3 protein, wherein the nucleotide sequence is codon optimised to express BDNF protein and/or NT-3 protein in a mammalian cell at a level that is higher relative to the level of protein expressed from a nucleotide sequence encoding the protein that is not codon optimised.

65. The method of claim 64, wherein the nucleotide sequence encoding human BDNF protein comprises SEQ ID NO: 2.

66. The method of claim 64, wherein the RNA molecule encodes BDNF and NT-3 and comprises a T2A element between BDNF and NT-3 for self-cleaving during protein translation.

67. The method of claim 64, wherein the nucleotide sequence encoding human NT-3 comprises SEQ ID NO: 4.

68. The method of claim 67, wherein the RNA molecule comprises the nucleotide sequence of SEQ ID NO: 15.

69. The method of claim 58, wherein the tissue is lining the perilymphatic compartments of a cochlea of a subject.

70. The method of claim 58, wherein the electric field is a non-uniform electric field that includes electric fields in the range of 1,000 μV/μm to 50,000 μV/μm.

71. The method of claim 58, wherein the electric potential difference is induced using a gene electrotransfer system comprising a controller configured to allow selective control of the pulse generator to control the sequence of pulses delivered via the electrodes, and wherein the controller is further configured to control electrical pulse parameters for the sequence of pulses based on carrier solution characteristics.

72. The method of claim 58, wherein the two or more electrodes are provided on a single elongate probe, the electrodes being contiguous with the probe arranged in line and relatively elongate along the direction of contiguity, wherein the electrode circumference is less than the electrode length.

73. The method of claim 58, wherein the two or more electrodes comprises an anode or anode array and a cathode or cathode array, and wherein the electric field is created between the anode or anode array and the cathode or cathode array in said target region for sufficient time to allow at least some of the RNA molecules to commit to enter the one or more cells.

74. The method of claim 73, wherein said anode or anode array and said cathode or cathode array are separated by between about 10 μm and about 5 mm.

75. The method of claim 72, wherein the total electric charge delivered during said electrotransfer is less than 5000 μCoulombs, optionally less than 500 μCoulombs, more optionally less than 1 μCoulombs.

76. The method of claim 72, wherein the electric charge is delivered through less than 100 electric pulses, optionally through 10 or fewer pulses, 5 or fewer pulses, 4 pulses, 3 pulses, 2 pulses or 1 pulse.

77. The method of claim 72, wherein each pulse is from about 100 us to about 500 ms in duration.

78. The method of claim 73, wherein each of said arrays independently comprises from 2 to 8 electrodes.

79. The method of claim 72, wherein the probe is shaped to model a cochlear implant prosthesis to be inserted into the cochlear for performing electrotransfer and subsequently removed.

80. The method of claim 58, wherein the electric field in induced using an electrotransfer system comprising:
an array of two or more linear electrodes with at least one insulating portion between, the electrodes being arranged in line and physically contiguous on a probe configured to be inserted into the tissue;
a pulse generator electrically connected to the electrodes of the array and configured to apply one or more electrical pulses to selectively drive the two or more electrodes as one or more anodes and one or more cathodes to generate an electric field in tissue adjacent the array,
wherein the electric field is shaped to provide controlled contours of gradients within the electric field based on the physical configuration of the electrodes, selection of electrodes and anodes and cathodes, and applied electrical pulse parameters; and
a controller configured to control the pulse generator, the controller being configured to control the pulse generator to apply a first sequence of one or more unipolar pulses using a first configuration of electrodes driven as anodes and cathodes to provide a first shaped electric field, and a second sequence of one or more unipolar pulses using a second configuration of electrodes driven as anodes and cathodes to provide a second shaped electric field, optionally wherein the respective lengths of the electrodes, and length of the insulating portion therebetween determines the pattern of the electric field generated adjacent the needle array when an electric pulse is applied to drive one or more adjacent electrodes at one end of the probe as an anode and one or more adjacent electrodes at an opposite end of the probe as a cathode.

81. The method of claim 58, wherein the electric field is induced using an electrotransfer system comprising:
at least one probe, each probe comprising:
a probe body;
a needle electrode array extending from the probe body configured as a needle to be inserted into tissue to be treated; and
a capacitive discharge circuit connected to the needle electrode array comprising capacitive charge storage configured to store a quantum of charge, and a switch actuatable to cause discharge of the stored quantum of charge through the needle electrode array,
the needle electrode array incorporating at least two electrodes, each electrode having a surface area substantially circumferential to the needle and exposed to directly contact tissue into which the needle electrode array is inserted, with an insulating section between neighbouring electrodes to form a contiguous linear array structure, the exposed surface area of each electrode being at a different distance from the needle electrode array tip, each electrode connected to the capacitive discharge circuit for driving as an anode or cathode during discharge of the quantum of charge, and wherein electrode length and length of the insulating section between neighbouring electrodes are configured to produce a target electric field shape in tissue adjacent to the array during discharge of the quantum of charge via the array, optionally wherein the electrotransfer system comprises a charging station having a DC power supply module, output terminals connectable to a probe to form electric contact to the capacitive discharge circuit, and charging control circuitry to control charging of the capacitive discharge circuit of the connected probe, and optionally wherein the needle electrode comprises:
a first needle which also acts as a first electrode;
a first concentric insulator sheathing the first needle to a predetermined first distance (L1) from the needle tip,
a concentric second electrode sheathing the first concentric insulator to a distance (L2) from a tipward end of the first concentric insulator, and
a second concentric insulator sheathing the second electrode to a distance (L3) from a tipward end of the second electrode, wherein the first needle and second concentric electrode are formed of conductive material and are electrically connected to positive and negative terminals of the capacitive discharge circuit, and optionally wherein the exposed length L1 of the first needle operates as a first electrode of a linear array, and the exposed length L3 of the second concentric electrode acts as the second electrode of a linear array.

82. A nucleic acid molecule comprising a nucleotide sequence selected from the group consisting of:
(a) a nucleotide sequence encoding human BDNF protein, wherein the nucleotide sequence is codon optimised to express BDNF protein in a mammalian cell at a level that is higher relative to the level of BDNF protein expressed from a nucleotide sequence encoding human BDNF protein that is not codon optimised; or
(b) the nucleotide sequence of (a) and a nucleotide sequence encoding human NT-3 protein, optionally wherein the human NT-3 protein is codon optimised to express the human NT-3 protein in the mammalian cell at a level that is higher relative to the level of NT-3 protein expressed from a nucleotide sequence encoding human NT-3 protein that is not codon optimised;
(c) a nucleotide sequence encoding human NT3 protein, wherein the nucleotide sequence is codon optimised to express NT3 protein in a mammalian cell at a level that is higher relative to the level of NT3 protein expressed from a nucleotide sequence encoding human NT3 protein that is not codon optimised;
(d) the nucleotide sequence of (c) and a nucleotide sequence encoding human BDNF protein, optionally wherein the human BDNF protein is codon optimised to express the human BDNF protein in the mammalian cell at a level that is higher relative to the level of BDNF protein expressed from a nucleotide sequence encoding human BDNF protein that is not codon optimised.

83. The nucleic acid molecule of claim 82, wherein the nucleotide sequence encoding human BDNF protein comprises SEQ ID NO: 2.

84. The nucleic acid molecule of claim 82, wherein the nucleotide sequence encoding human NT-3 comprises SEQ ID NO: 4.

85. The nucleic acid molecule of claim 82, wherein the nucleotide sequence encoding the BDNF and NT-3 proteins is codon optimised to express each protein in a mammalian cell at a level that is higher relative to the level of protein expressed from a nucleotide sequence encoding the protein that is not codon optimised.

86. The nucleic acid of claim 82, wherein the nucleic acid is mRNA.

87. The nucleic acid of claim 82, wherein the nucleic acid comprises the nucleotide sequence of SEQ ID NO: 15.

88. A composition comprising the nucleic acid molecule of claim 82, and a pharmaceutically acceptable carrier.

89. The composition of claim 88, wherein the pharmaceutically acceptable carrier has low electrical conductivity.

90. The composition of claim 89, wherein the pharmaceutically acceptable carrier is iso-osmotic with respect to extracellular fluid.

91. The composition of claim 88, wherein the pharmaceutically acceptable carrier comprises one or more polysaccharoses, optionally wherein the polysaccharose is sucrose, and optionally wherein the sucrose is at a concentration in the range of from about 5-15% w/v, typically 6-14% w/v, 7-13% w/v, 8-12% w/v, 9-12% w/v, or 9-11% w/v.

92. The composition of claim 88, wherein the pharmaceutically acceptable carrier comprises a low conductivity radio-opaque agent suitable for imaging dispersion of the nucleic acid molecules within the target tissue.

93. A kit comprising:
at least one packaged dose of a composition as claimed in claim 88; and
an electrotransfer system comprising:
at least one probe, each probe comprising:
a probe body;
a needle electrode array extending from the probe body configured as a needle to be inserted into tissue to be treated; and
a capacitive discharge circuit connected to the needle electrode array comprising capacitive charge storage configured to store a quantum of charge, and a switch actuatable to cause discharge of the stored quantum of charge through the needle electrode array,
the needle electrode array incorporating at least two electrodes, each electrode having a surface area substantially circumferential to the needle and exposed to directly contact tissue into which the needle electrode array is inserted, with an insulating section between neighbouring electrodes to form a contiguous linear array structure, the exposed surface area of each electrode being at a different distance from the needle electrode array tip, each electrode connected to the capacitive discharge circuit for driving as an anode or cathode during discharge of the quantum of charge, and wherein electrode length and length of the insulating section between neighbouring electrodes are configured to produce a target electric field shape in tissue adjacent to the array during discharge of the quantum of charge via the array.
